(19) United States
(12) Patent Application Publication
(10) Pub. No.: US 2007/0006396 A9
(43) Pub. Date: Jan. 11, 2007

(54) Composition for dyeing keratin fibers with a cationic direct dye and a thickening polymer

(75) Inventor: Christine Rondeau, Sartrouville (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL S.A.

(21) Appl. No.: 11/087,013

(22) Filed: Mar. 23, 2005

Related U.S. Application Data
(63) Continuation of application No. 09/349,105, filed on Jul. 8, 1999, now abandoned.

(30) Foreign Application Priority Data
Jul. 9, 1998 (FR) 98 08833

(51) Int. Cl. A61K 7/13

(52) U.S. Cl. 8/405

(57) ABSTRACT

The invention relates to a composition for dyeing keratin fibers, in particular human keratin fibers such as the hair, comprising, in a medium which is suitable for dyeing, at least one cationic direct dye of given formula, and which is characterized in that it also contains at least one thickening polymer comprising at least one sugar unit. The invention also relates to the dyeing processes and dyeing devices using it.

The invention relates to a composition for dyeing keratin fibers, in particular human keratin fibers such as the hair, comprising, in a medium which is suitable for dyeing, at least one cationic direct dye of given formula and at least one thickening polymer comprising at least one sugar unit.

The invention also relates to the dyeing processes and dyeing devices using the composition.

Two types of dyeing may be distinguished in the haircare sector.

The first is semi-permanent or temporary dyeing, or direct dyeing, which uses dyes capable of giving the hair a natural coloration, a more or less pronounced color change which may withstand shampooing several times. These dyes are also known as direct dyes; they can be used with or without an oxidizing agent. In the presence of an oxidizing agent, the aim is to obtain lightening dyeing. Lightening dyeing is carried out by applying a mixture, prepared at the time of use, of a direct dye and an oxidizing agent to the hair, and makes it possible in particular to obtain, by lightening the melanin in the hair, an advantageous effect such as a unified color in the case of grey hair, or to bring out the color in the case of naturally pigmented hair.

The second is permanent dyeing or oxidation dyeing. This is carried out with so-called “oxidation” dyes comprising oxidation dye precursors and couplers. Oxidation dye precursors, commonly known as “oxidation bases”, are compounds which are initially colorless or weakly colored which develop their dyeing power on the hair in the presence of oxidizing agents added at the time of use, leading to the formation of colored compounds and dyes. The formation of these colored compounds and dyes results either from an oxidative condensation of the “oxidation bases” with themselves or from an oxidative condensation of the oxidation bases with coloration-modifying compounds commonly known as “couplers”, which are generally present in the dye compositions used in oxidation dyeing.

It is known practice to add direct dyes to oxidation dyes in order to vary—the shades obtained with the said oxidation dyes or to enrich the shades with glints.

Among the cationic direct dyes available in the sector of dyeing keratin fibers, in particular human keratin fibers, the compounds whose structure is developed in the text hereinbelow are already known; nevertheless, these dyes lead to colorations which have characteristics that could still be improved, such as the intensity, the homogeneity of the color distributed along the fiber, in which case the coloration is said to be too selective, and the staying power, in terms of the resistance to the various attacking factors to which the hair may be subjected (light, bad weather, shampooing).

After considerable research conducted in this matter, the inventor has now discovered that it is possible to obtain novel compositions for dyeing keratin fibers which are capable of giving more intense and yet unselective colorations which show good resistance to the various attacking factors to which the hair may be subjected, by combining at least one thickening polymer comprising at least one sugar unit with at least one known cationic direct dye of the prior art, which have the respective formulae defined below.

This discovery forms the basis of the present invention.

A first subject of the present invention is thus a composition for dyeing keratin fibers, and in particular human keratin fibers such as the hair, containing, in a medium which is suitable for dyeing, (i) at least one cationic direct dye whose structure corresponds to formulae (I) to (III′) defined below, characterized in that it also contains (ii) at least one thickening polymer comprising at least one sugar unit.

(i) The cationic direct dye which can be used according to the present invention is a compound chosen from those of formulae (I), (II), (III) and (III′) below:

a) the compounds of formula (I) below: 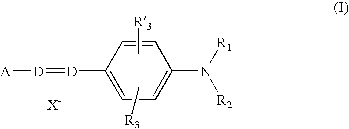
in which:
D represents a nitrogen atom or a —CH group,
R₁ and R₂, which may be identical or different, represent a hydrogen atom; a C₁-C₄ alkyl radical which can be substituted with a —CN, —OH or —NH₂ radical or form, with each other or a carbon atom of the benzene ring of formula (I), a heterocycle optionally containing oxygen or nitrogen, which can be substituted with one or more C₁-C₄ alkyl radicals; a 4′-aminophenyl radical,
R₃ and R′₃, which may be identical or different, represent a hydrogen atom, a halogen atom chosen from chlorine, bromine, iodine and fluorine, a cyano radical, or a C₁-C₄ alkyl, C₁-C₄ alkoxy or acetyloxy radical,
X⁻ represents an anion preferably chosen from chloride, methyl sulphate, perchlorate and acetate,
A represents a group chosen from the structures A1 to A19 below: 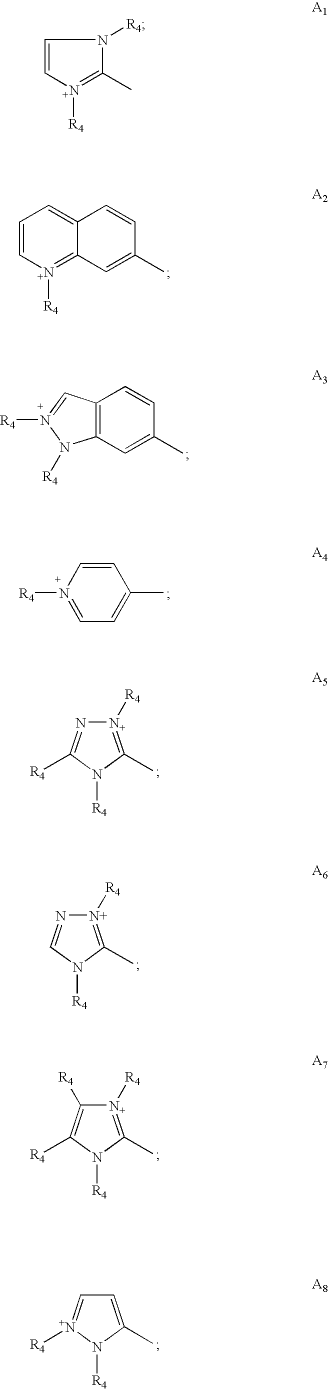 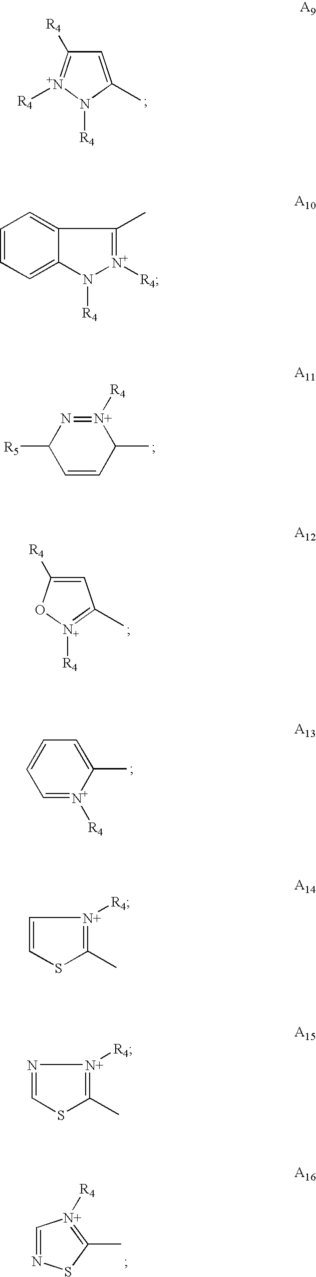 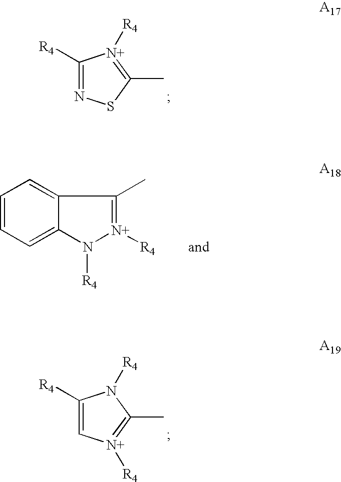
   in which R₄ represents a C₁-C₄ alkyl radical which can be substituted with a hydroxyl radical and R₅ represents a C₁-C₄ alkoxy radical, with the proviso that when D represents —CH, when A represents A₄ or A₁₃ and when R₃ is other than an alkoxy radical, then R₁ and R₂ do not simultaneously denote a hydrogen atom;

b) the compounds of formula (II) below: 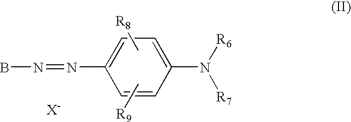
in which:
R₆ represents a hydrogen atom or a C₁-C₄ alkyl radical,
R₇ represents a hydrogen atom, an alkyl radical which can be substituted with a —CN radical or with an amino group, a 4′-aminophenyl radical or forms with R₆ a heterocycle optionally containing oxygen and/or nitrogen, which can be substituted with a C₁-C₄ alkyl radical,
R₈ and R₉, which may be identical or different, represent a hydrogen atom, a halogen atom such as bromine, chlorine, iodine or fluorine, a C₁-C₄ alkyl or C₁-C₄ alkoxy radical or a —CN radical,
X⁻ represents an anion preferably chosen from chloride, methyl sulphate, and acetate,
B represents a group chosen from the structures B₁ to B₆ below: 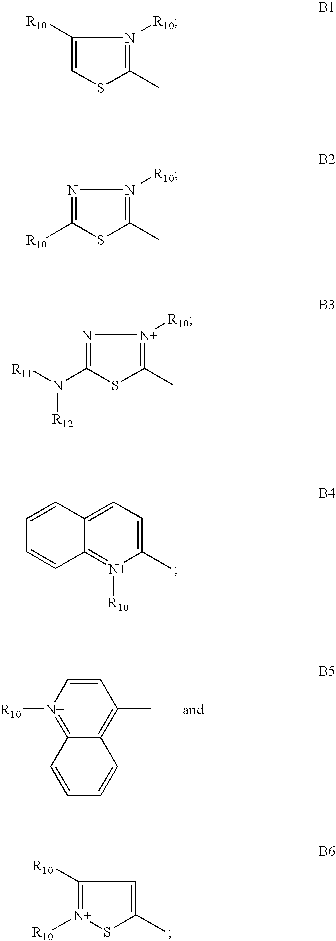
   in, which R₁₀ represents a C₁-C₄ alkyl radical, R₁₁ and R₁₂, which may be identical or different, represent a hydrogen atom or a C₁-C₄ alkyl radical;

c) the compounds of formulae (III) and (III′) below: 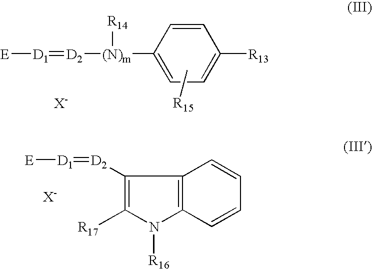
in which:
R₁₃ represents a hydrogen atom, a C₁-C₄ alkoxy radical, a halogen atom such as bromine, chlorine, iodine or fluorine, or an amino radical,
R₁₄ represents a hydrogen atom, a C₁-C₄ alkyl radical or forms, with a carbon atom of the benzene ring, a heterocycle optionally containing oxygen and/or substituted with one or more C₁-C₄ alkyl groups,
R₁₅ represents a hydrogen atom or a halogen atom such as bromine, chlorine, iodine or fluorine,
R₁₆ and R₁₇, which may be identical or different, represent a hydrogen atom or a C₁-C₄ alkyl radical,
D₁ and D₂, which may be identical or different, represent a nitrogen atom or a —CH group,
m=0 or 1,
it being understood that when R₁₃ represents an unsubstituted amino group, then D₁ and D₂ simultaneously represent a —CH group and m=0,
X⁻ represents an anion preferably chosen from chloride, methyl sulphate and acetate,
E represents a group chosen from the structures E₁ to E₈ below: 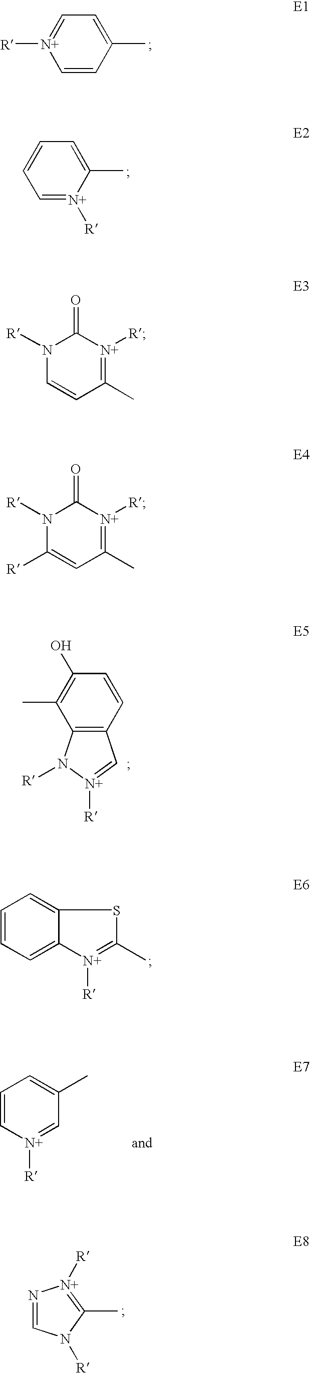
   in which R′ represents a C₁-C₄ alkyl radical;
   when m=0 and when D₁ represents a nitrogen atom, then E can also denote a group of structure E9 below: 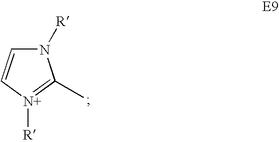
   in which R′ represents a C₁-C₄ alkyl radical.

In the structures (I) to (III) and (III′) defined above, the C₁-C₄ alkyl or alkoxy group preferably denotes methyl, ethyl, butyl, methoxy or ethoxy.

The cationic direct dyes of formulae (I), (II), (III) and (III′) which can be used in the dye compositions in accordance with the invention are known compounds and are described, for example, in patent applications WO 95/01772, WO 95/15144 and EP-A-0,714,954, the disclosure of each of which is hereby specifically incorporated herein by reference.

Among the cationic direct dyes of formula (I) which can be used in the dye compositions in accordance with the invention, mention may be made more particularly of the compounds corresponding to the structures (I1) to (I54) below:

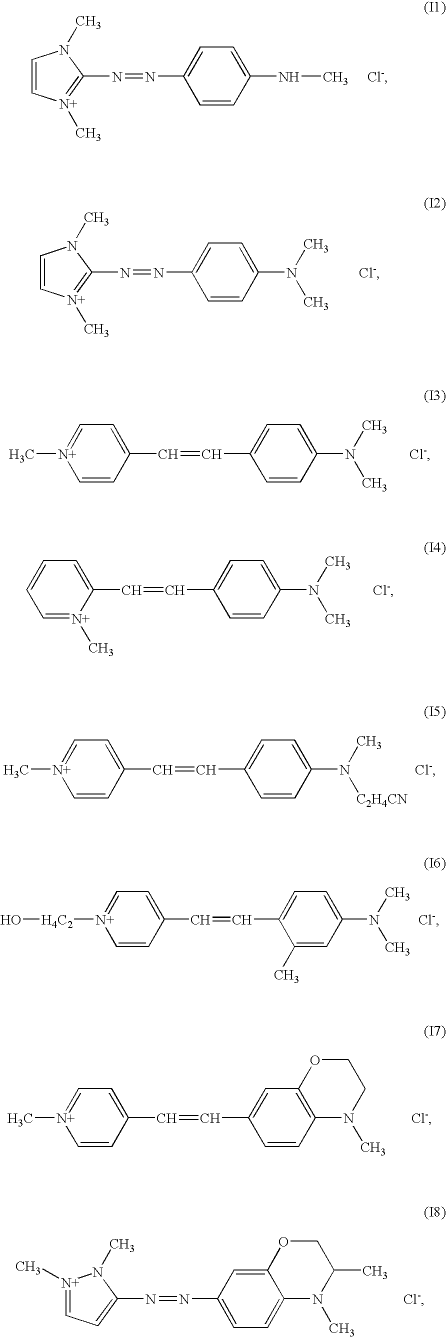

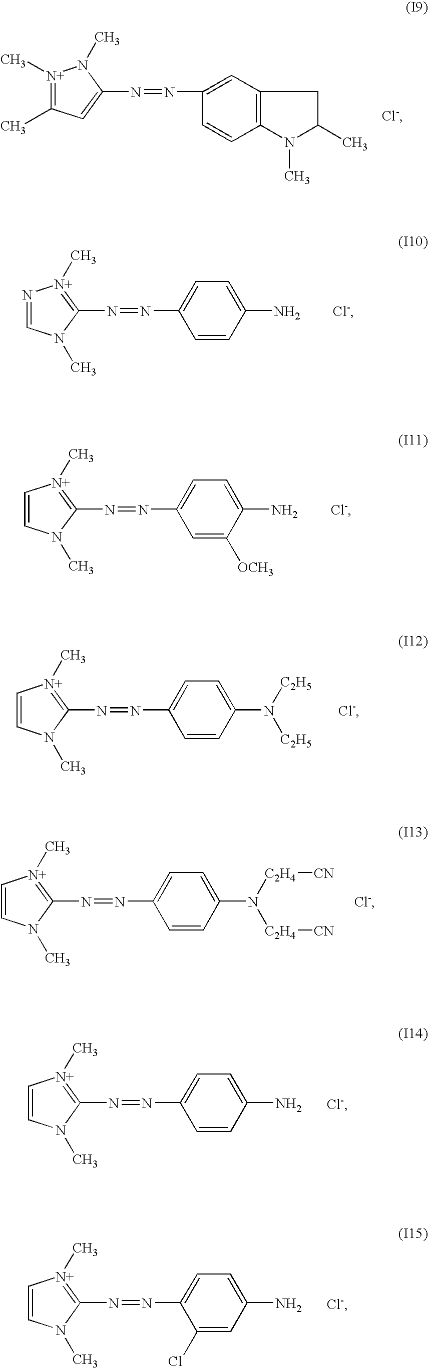

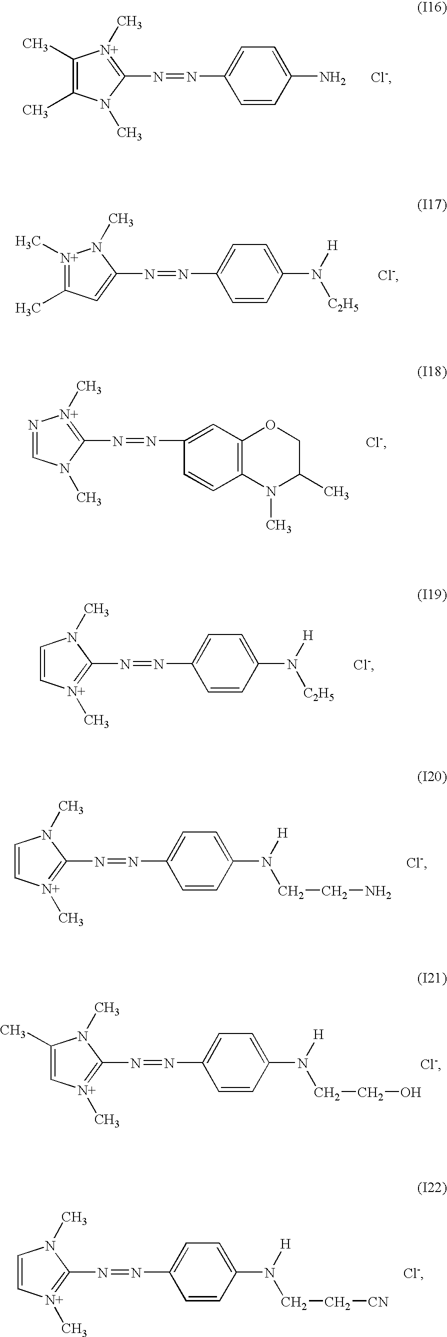

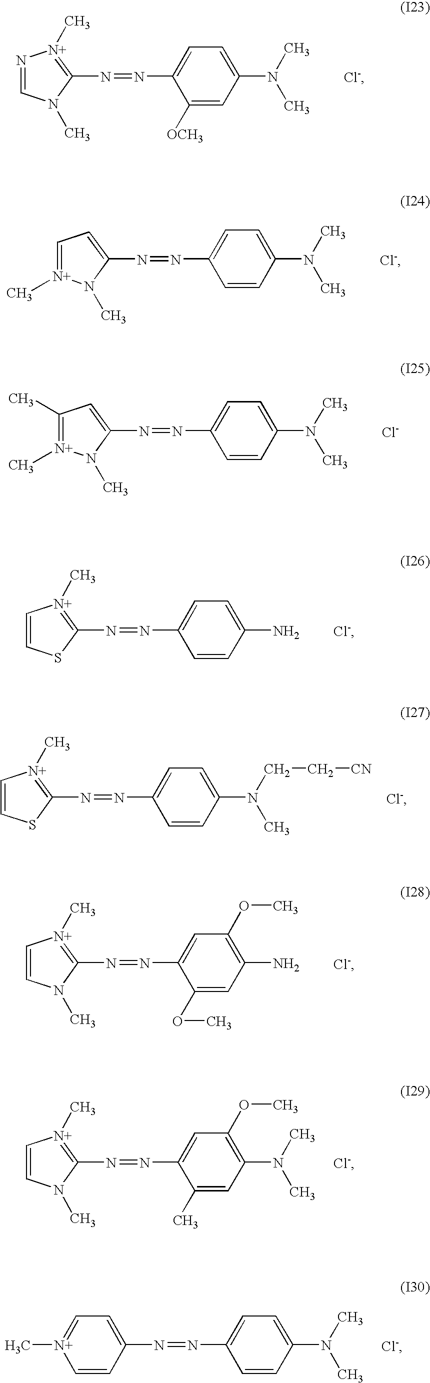

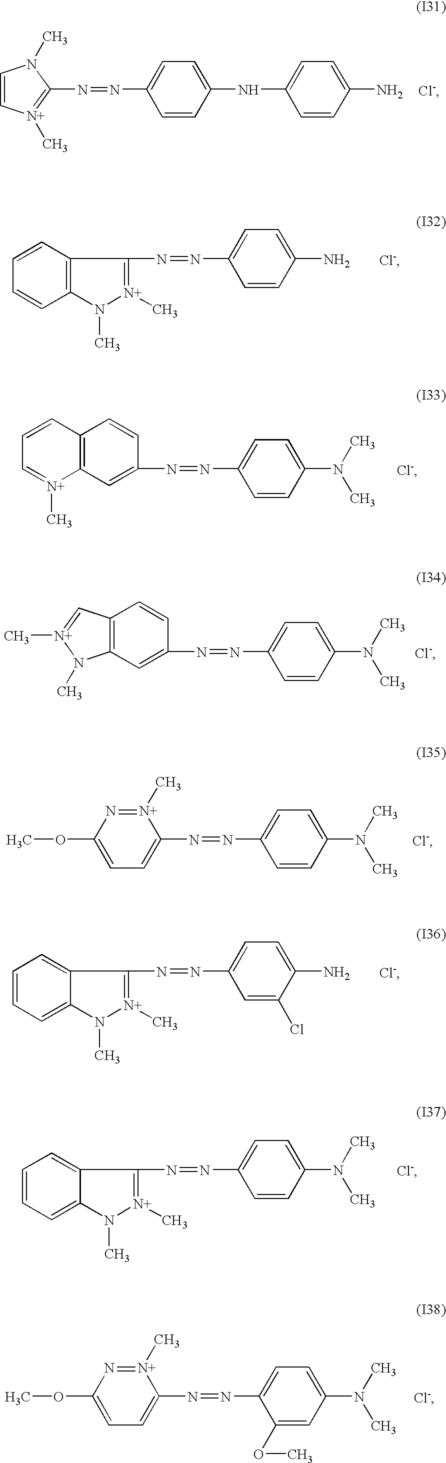

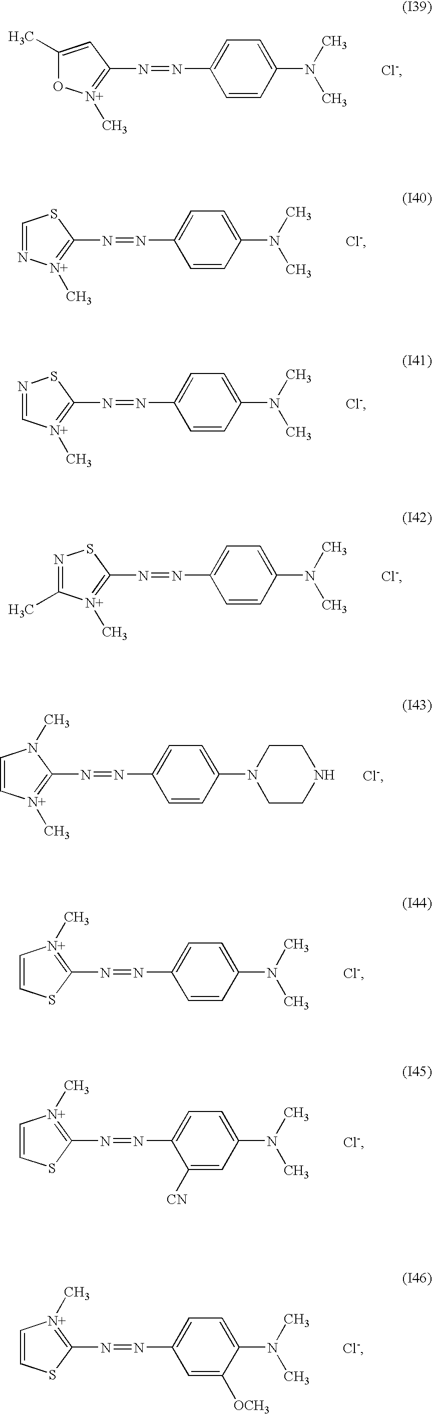

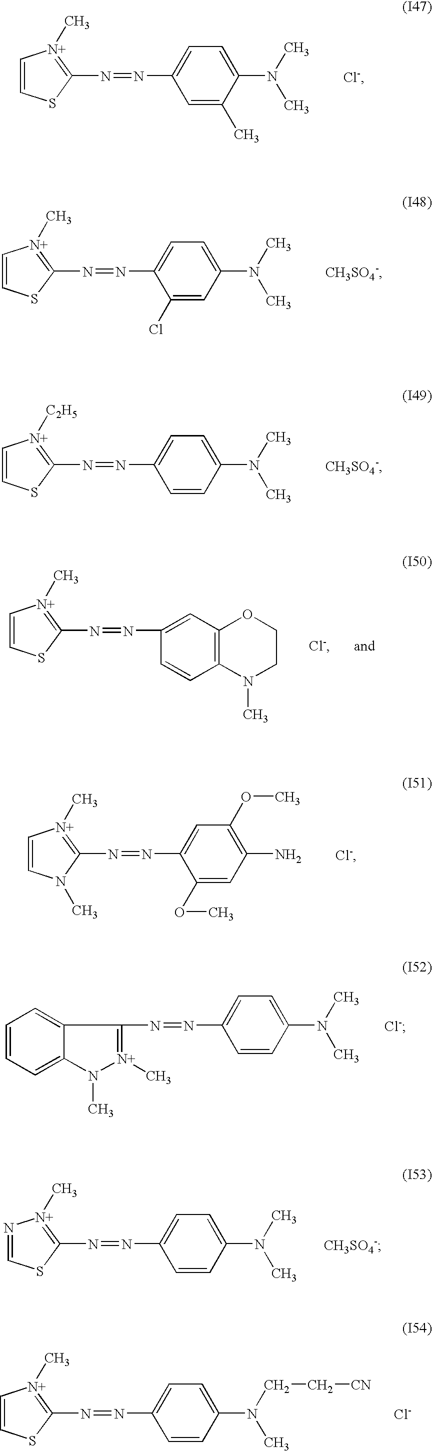

Among the compounds of structures (I1) to (I54) described above, the ones most particularly preferred are the compounds corresponding to the structures (I1), (I2), (I14) and (I31).

Among the cationic direct dyes of formula (II) which can be used in the dye compositions in accordance with the invention, mention may be made more particularly of the compounds corresponding to the structures (II1) to (II9) below:

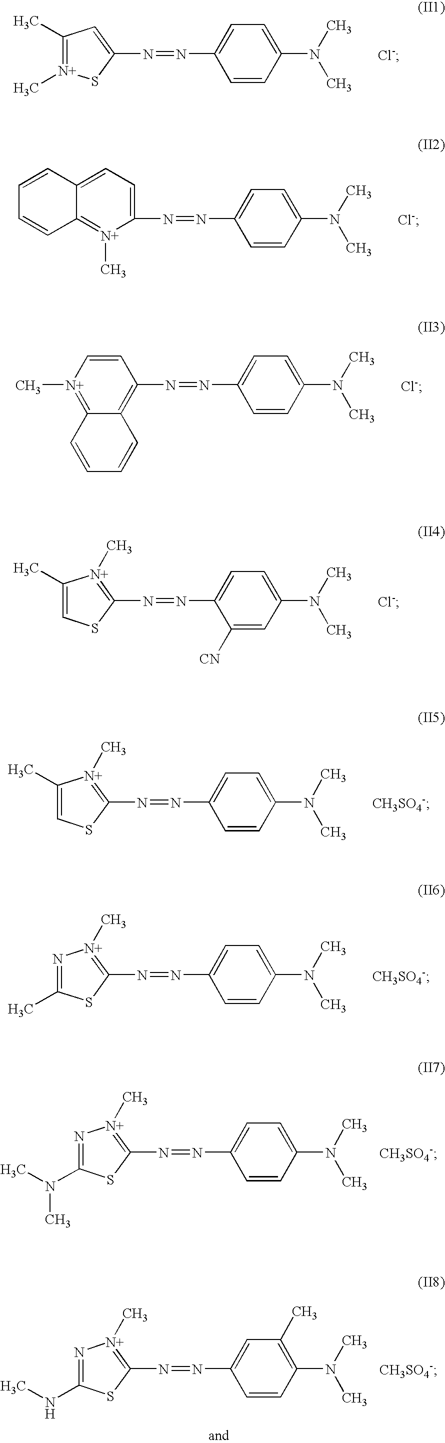

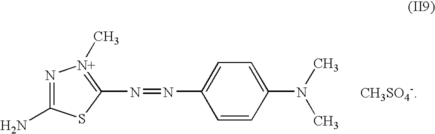

Among the cationic direct dyes of formula (III) which can be used in the dye compositions in accordance with the invention, mention may be made more particularly of the compounds corresponding to the structures (III1) to (III18) below:

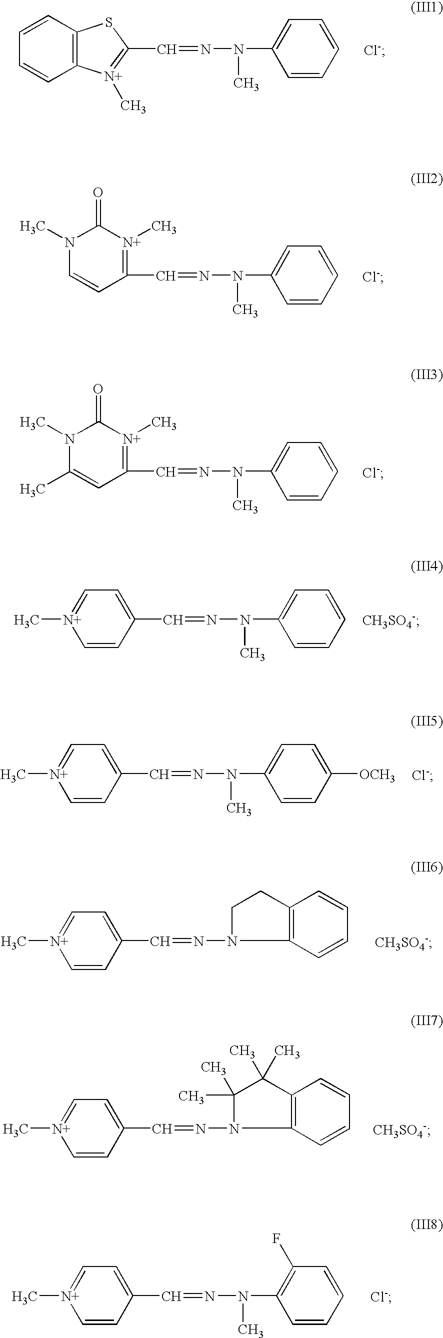

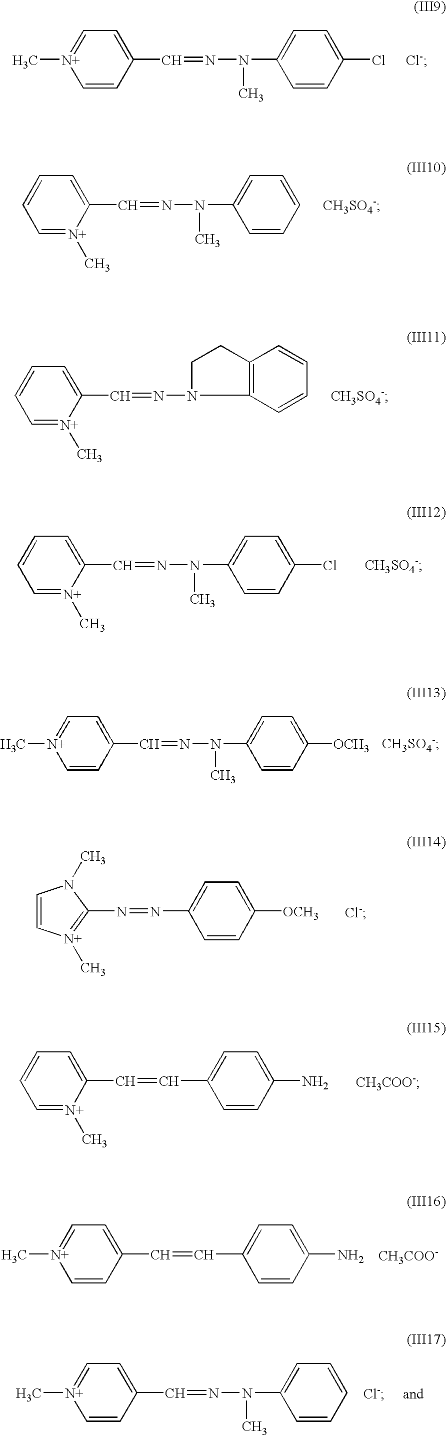

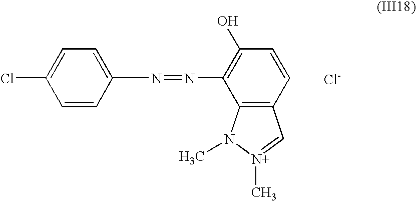

Among the specific compounds of structures (III1) to (III18) described above, the ones most particularly preferred are the compounds corresponding to the structures (III4), (III5) and (III13).

Among the cationic direct dyes of formula (III′) which can be used in the dye compositions in accordance with the invention, mention may be made more particularly of the compounds corresponding to the structures (III′1) to (III′3) below:

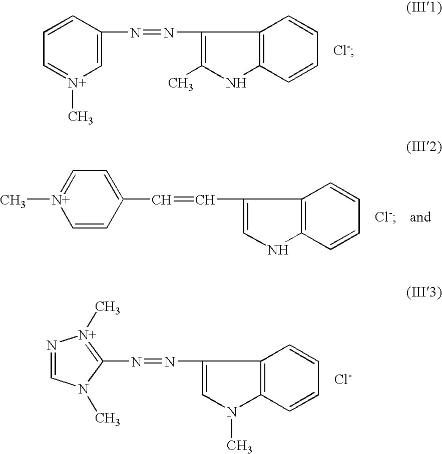

The cationic direct dye(s) used according to the invention preferably represent(s) from 0.001 to 10% by weight approximately relative to the total weight of the dye composition and even more preferably from 0.005 to 5% by weight approximately relative to this weight.

(ii) The thickening polymer comprising at least one sugar unit which can be used according to the present invention is chosen from:

(ii)₁—nonionic guar gums;
(ii)₂—biopolysaccharide gums of microbial origin, such as scleroglucan or xanthan gums;
(ii)₃—gums derived from plant exudates, such as gum arabic, ghatti gum, karaya gum, gum tragacanth, carrageenan gum, agar gum and carob gum;
(ii)₄—pectins;
(ii)₅—alginates;
(ii)₆—starches; and
(ii)₇—hydroxyalkylcelluloses and carboxyalkylcelluloses.

For the purposes of the present invention, the expression “sugar unit” denotes a monosaccharide portion (i.e., monosaccharide or oside or simple sugar) or an oligosaccharide portion (short chains formed from the linking of monosaccharide units, which may be different) or a polysaccharide portion (long chains consisting of monosaccharide units, which may be different, i.e., polyholosides or polyosides (homopolyosides or heteropolyosides). The saccharide units can also be substituted with alkyl, hydroxyalkyl, alkoxy, acyloxy or carboxyl groups.

The nonionic guar gums can be modified or unmodified. The unmodified guar gums are, for example, the products sold under the name Vidogum GH 175 by the company Unipectine and under the name Jaguar C by the company Meyhall.

According to the present invention, it is preferred to use nonionic guar gums modified with C₁-C₆ hydroxyalkyl groups.

Among the hydroxyalkyl groups which may be mentioned, for example, are hydroxymethyl, hydroxyethyl, hydroxypropyl and hydroxybutyl groups. These guar gums are well known in the state of the art and can be prepared, for example, by reacting the corresponding alkene oxides such as, for example, propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, preferably ranges from 0.4 to 1.2.

Such nonionic guar gums optionally modified with hydroxyalkyl groups are sold, for example, under the trade names Jaguar HP8, Jaguap HP60 and Jaguar HP120, Jaguar DC 293 and Jaguar HP 105 by the company Rhône-Poulenc (Meyhall) or under the name Galactasol 4H4FD2 by the company Aqualon.

The biopolysaccharide gums of microbial origin, such as the scleroglucan or xanthan gums, the gums derived from plant exudates such as gum arabic, ghatti gum, karaya gum, gum tragacanth, carrageenan gum, agar gum and carob gum, the hydroxyalkylcelluloses and carboxymethylcelluloses, pectins, alginates and starches are well known to those skilled in the art and are described in particular in the book by Robert L. Davidson entitled “Handbook of Water soluble gums and resins” published by McGraw Hill Book Company (1980), the disclosure of which is specifically incorporated by reference.

Among these gums, the scleroglucans more particularly used according to the present invention are represented by the products sold under the name Actigum CS by the company Sanofi Bio Industries and in particular Actigum CS 11, and under the name Amigel by the company Alban Muller International. Other scleroglucans, such as the one treated with glyoxal in French patent application No. 2,633,940, can also be used, the disclosure of which is incorporated by reference.

The xanthan gums more particularly used according to the present invention are represented by the products sold under the names Keltrol, Keltrol T, Keltrof T F, Keltrol B T, Keltrol R D and Keltrol C G by the company Nutrasweet Kelco, or under the names Rhodicare S and Rhodicare H by the company Rhodia Chimie.

The hydroxyalkylcelluloses are more particularly hydroxyethylcelluloses, such as those sold under the names Cellosize QP3L, Cellosize QP4400H, Cellosize QP30000H, Cellosize HEC30000A and Cellosize Polymer PCG10 by the company Amerchol, or Natrosol 250HHR, Natrosol 250 MR, Natrosol 250M, Natrosol 250HHXR, Natrosol 250HHX, Natrosol 250HR and Natrosol HX by the company Hercules, or Tylose H1000 by the company Hoechst.

The hydroxyalkylcelluloses are also, more particularly, hydroxypropylcelluloses such as the products sold under the names Klucel E F, Klucel H, Klucel L H F, Klucel M F and Klucel G by the company Aqualon.

Among the carboxyalkylcelluloses preferably used is carboxymethylcellulose, for which mention may be made of the products sold under the names Blanose 7M8/SF, Blanose Raffinée 7M, Blanose 7LF, Blanose 7MF, Blanose 9M31F, Blanose 12M31XP, Blanose 12M31P, Blanose 9M31XF, Blanose 7H, Blanose 7M31 and Blanose 7H3SXF by the company Aqualon, or Aquasorb A500 and Ambergum 1221 by the company Hercules, or Cellogen HP810A and Cellogen HP6HS9 by the company Montello, or Primellose by the company Avebe.

The thickening polymers (ii) used in the compositions of the present invention are preferably present in a proportion of from 0.01 to 10% by weight approximately, in particular in a proportion of from 0.1 to 5% by weight approximately, relative to the total weight of the dye composition applied to the keratin fibers.

The medium which is suitable for dyeing (or support) generally comprises water or a mixture of water and at least one organic solvent to dissolve the compounds which would not be sufficiently water-soluble. As organic solvents, mention may be made, for example, of C₁-C₄ lower alkanols such as ethanol and isopropanol; aromatic alcohols such as benzyl alcohol, as well as similar products and mixtures thereof.

The solvents can be present in proportions preferably from 1 to 40% by weight approximately relative to the total weight of the dye composition, and even more preferably from 5 to 30% by weight approximately.

The pH of the dye composition in accordance with the invention is generally approximately from 2 to 11 and preferably approximately from 5 to 10. It can be adjusted to the desired value using acidifying or basifying agents usually used for dyeing keratin fibers.

Among the acidifying agents, mention may be made, by way of example, of inorganic or organic acids such as hydrochloric acid, orthophosphoric acid, sulphuric acid, carboxylic acids such as acetic acid, tartaric acid, citric acid and lactic acid, and sulphonic acids.

Among the basifying agents, mention may be made, by way of example, of aqueous ammonia, alkaline carbonates, alkanolamines such as mono-, di- and triethanolamine and derivatives thereof, sodium hydroxide, potassium hydroxide and the compounds of formula (IV) below: 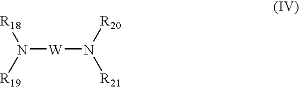
in which W is a propylene residue optionally substituted with a hydroxyl group or a C₁-C₆ alkyl radical; R₁₈, R₁₉, R₂₀ and R₂₁, which may be identical or different, represent a hydrogen atom or a C₁-C₆ alkyl or C₁-C₆ hydroxyalkyl radical.

In addition to the cationic direct dye(s) (i) defined above, the dye composition in accordance with the invention can comprise one or more additional direct dyes which can be chosen, for example, from nitrobenzene dyes, anthraquinone dyes, naphthoquinone dyes, triarylmethane dyes, xanthene dyes and azo dyes which are non-cationic.

When it is intended for oxidation dyeing, the dye composition in accordance with the invention comprises, in addition to the cationic direct dye(s) (i), one or more oxidation bases chosen from the oxidation bases conventionally used for oxidation dyeing and among which mention may be made in particular of para-phenylenediamines, bis(phenyl)alkylenediamines, para-aminophenols, ortho-aminophenols and heterocyclic bases.

When they are used, the oxidation base(s) preferably represent(s) from 0.0005 to 12% by weight approximately relative to the total weight of the dye composition, and even more preferably from 0.005 to 6% by weight approximately relative to this weight.

When it is intended for oxidation dyeing, the dye composition in accordance with the invention can also comprise, in addition to the cationic direct dye (i) and the thickening polymer (ii) as well as the oxidation bases, one or more couplers so as to modify the shades obtained or to enrich them with glints, by using the cationic direct dye(s) (i) and the oxidation base(s).

The couplers which can be used in the dye composition in accordance with the invention can be chosen from the couplers used conventionally in oxidation dyeing and among which mention may be made in particular of meta-phenylenediamines, meta-aminophenols, meta-diphenols and heterocyclic couplers.

When it is (they are) present, the coupler(s) preferably represent(s) from 0.0001 to 10% by weight approximately relative to the total weight of the dye composition, and even more preferably from 0.005 to 5% by weight approximately relative to this weight.

The dye composition in accordance with the invention can also contain various adjuvants conventionally used in compositions for dyeing the hair, such as antioxidants, penetrating agents, sequestering agents, fragrances, buffers, dispersing agents, surfactants, film-forming agents, ceramides, preserving agents, screening agents, such as sunscreens, and opacifiers.

Needless to say, a person skilled in the art will take care to select this (these) optional complementary compound(s) such that the advantageous properties intrinsically associated with the dye composition in accordance with the invention are not, or are not substantially, adversely affected by the addition(s) envisaged.

The dye composition according to the invention can be in various forms, such as in the form of liquids, shampoos, creams or gels or any other form which is suitable for dyeing keratin fibers, and in particular human hair. It can be obtained by mixing, at the time of use, a composition, which may be pulverulent, containing the cationic direct dye(s) with a composition containing the thickening polymer (ii) according to the invention.

When the combination of the cationic direct dye (i) and the thickening polymer (ii) according to the invention is used in a composition intended for oxidation dyeing (in which case one or more oxidation bases are used, optionally in the presence of one or more couplers) or when it is used in a composition intended for lightening direct dyeing, then the dye composition in accordance with the invention also comprises at least one oxidizing agent chosen, for example, from hydrogen peroxide, urea peroxide, alkali metal bromates, persalts such as perborates and persulphates, and enzymes such as peroxidases, lactases and two-electron oxidoreductases. It is particularly preferred to use hydrogen peroxide or enzymes.

Another subject of the invention is a process for dyeing keratin fibers, and in particular human keratin fibers such as the hair, using the dye composition as defined above.

According to a first variant of this dyeing process in accordance with the invention, at least one dye composition as defined above is applied to the fibers, for a period which is sufficient to develop the desired coloration, after which the fibers are rinsed, optionally washed with shampoo, rinsed again and dried.

The time required to develop the coloration on the keratin fibers is generally from 3 to 60 minutes and even more specifically from 5 to 40 minutes.

According to a second variant of this dyeing process in accordance with the invention, at least one dye composition as defined above is applied to the fibers, for a period which is sufficient to develop the desired coloration, without final rinsing.

According to one specific embodiment of this dyeing process, and when the dye composition in accordance with the invention comprises at least one oxidation base and at least one oxidizing agent, the dyeing process comprises a first step which comprises separately storing, on the one hand, a composition (A1) comprising, in a medium which is suitable for dyeing, at least one cationic direct dye (i) as defined above and at least one oxidation base, and, on the other hand, a composition (B1) comprising, in a medium which is suitable for dyeing, at least one oxidizing agent, and then in mixing them together at the time of use, after which this mixture is applied to the keratin fibers, the composition (A1) or the composition (B1) containing the thickening polymer (ii) as defined above.

According to another specific embodiment of this dyeing process, and when the dye composition in accordance with the invention comprises at least one oxidizing agent, the dyeing process comprises a first step which consists in separately storing, on the one hand, a composition (A2) comprising, in a medium which is suitable for dyeing, at least one cationic direct dye (i) as defined above, and, on the other hand, a composition (B2) comprising, in a medium which is suitable for dyeing, at least one oxidizing agent, and then in mixing them together at the time of use, after which this mixture is applied to the keratin fibers, the composition (A2) or the composition (B2) containing the thickening polymer as defined above.

Another subject of the invention is a multi-compartment dyeing device or dyeing “kit” or any other multi-compartment packaging system, a first compartment of which comprises the composition (A1) or (A2) as defined above and a second compartment of which comprises the composition (B1) or (B2) as defined above. These devices can be equipped with means for dispensing the desired mixture onto the hair, such as the devices described in patent FR 2,586,913, the disclosure of which is specifically incorporated by reference.

The examples which follow are intended to illustrate the invention without, however, limiting its scope.

### EXAMPLES

### Examples 1 to 4

The four direct dyeing, compositions given in the table below were prepared:

(all contents expressed in grams)

| | Ex- | | | Ex- |
|---|---|---|---|---|
| | ample 1 | Example 2 | Example 3 | ample 4 |
| Cationic direct dye of formula | 0.2 | | | |
| (I1) | | | | |
| Cationic direct dye of formula | | 0.2 | | |
| (I2) | | | | |
| Cationic direct dye of formula | | | 0.2 | |
| (I14) | | | | |
| Cationic direct dye of formula | | | | 0.15 |
| (I31) | | | | |
| Hydroxyethylcellulose sold | 1.0 | | | |
| under the name Natrosol 250 | AM* | | | |
| HHR by the company | | | | |
| Aqualon | | | | |
| Carboxymethylcellulose sold | | 1.0 AM* | | |
| under the name Blanose 7M | | | | |
| by the company Aqualon | | | | |
| Guar gum sold under the | | | 1.0 AM* | |
| name Vidogum GH175 by the | | | | |
| company Unipectine | | | | |
| Scleroglucan gum sold under | | | | 1.0 |
| the name Amigel by the | | | | AM* |
| company Alban Muller | | | | |
| International | | | | |
| Ethanol | 10 | 10 | 10 | 10 |
| 2-Amino-2-methyl-1-propanol | pH 9 | pH 9 | pH 9 | pH 9 |
| qs | | | | |
| Demineralized water qs | 100 | 100 | 100 | 100 |

AM* denotes active material
AM* denotes active material

The above compositions were each applied for 30 minutes to locks of natural grey hair containing 90% white hairs. The locks of hair were then rinsed, washed with a standard shampoo and then dried.

The locks were dyed in the following shades:

| Examples | Shades obtained |
|---|---|
| 1 | Bright red |
| 2 | Bright red |
| 3 | Bright orange |
| 4 | Bright purple |

1-55. (canceled)

56. A ready-to-use composition for dyeing keratin fibers, comprising:
(i) at least one cationic direct dye chosen from compounds of formula (I), (II), (III) and (III′) below, and
(ii) at least one thickening polymer;
(a) wherein said compounds of formula (I) are chosen from compounds of formula: 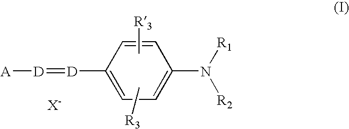
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ may form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 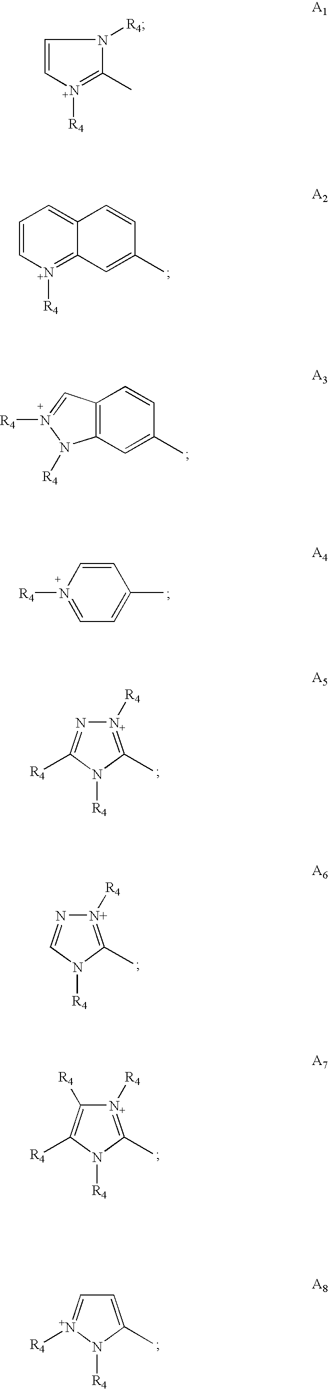 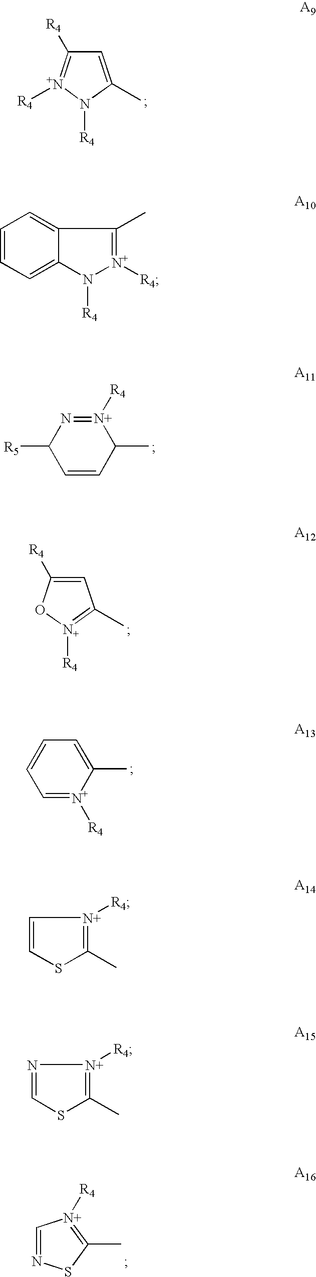 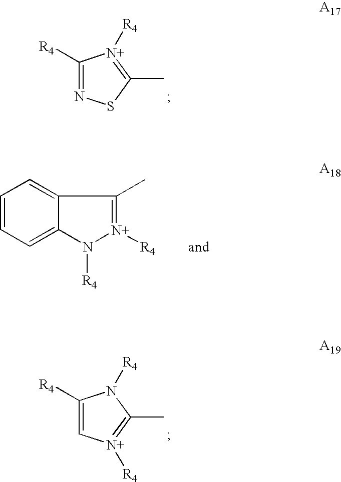
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 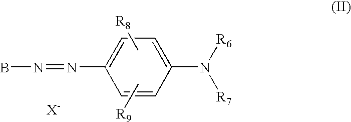
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals, C₁-C₄ alkoxy radicals and a —CN radical,
X⁻ is chosen from anions,
B is chosen from structures B₁ to B₆ below: 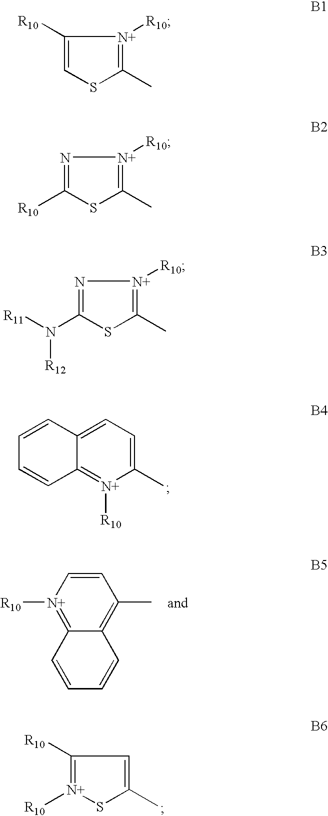
in which:
R₁₀ is chosen from C₁-C₄ alkyl radicals, and
R₁₁, and R₁₂, which may be identical or different, are chosen from a hydrogen atom and C₁-C₄ alkyl radicals;
(c) wherein said compounds of formula (III) and (III′) are chosen from compounds of formula: 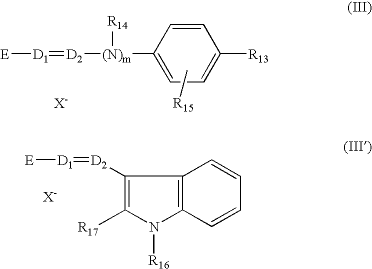
in which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 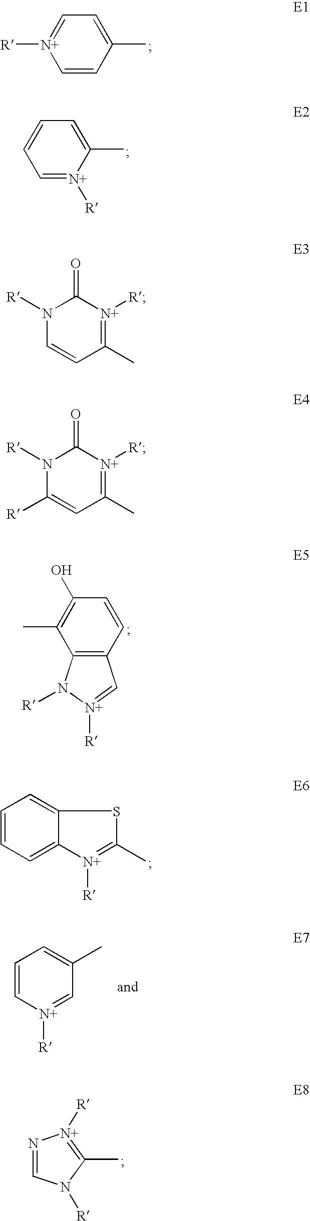
in which R′ is chosen from C₁-C₄ alkyl radicals;
wherein when m is 0 and when D₁ represents a nitrogen atom, E can be further chosen from structure E9 below: 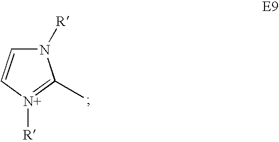
in which R′ is chosen from C₁-C₄ alkyl radicals; and
(d) wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses,
with the provisos that
(1) when said at least one cationic direct dye is chosen from compounds of formula (I) wherein:
both D's are simultaneously nitrogen atoms,
R₃ and R′₃ are simultaneously hydrogen atoms,
R₁ and R₂ are simultaneously unsubstituted methyl radicals, and
A is A₆ wherein R₄ is an unsubstituted methyl radical, or
(2) when said at least one cationic direct dye is chosen from compounds of formula (III) wherein:
D₁ and D₂ are simultaneously nitrogen atoms,
m is zero,
R₁₅ is a hydrogen atom,
R₁₃ is a dimethylamino radical, and
E is E₈ wherein R′ is an unsubstituted methyl group,
then the at least one thickening polymer is not chosen from at least one nonionic guar gum; and
with the further provisos that
(1) when said at least one cationic direct dye is chosen from compounds of formula (I) wherein:
both D's are simultaneously nitrogen atoms, and
A is chosen from A₄ and A₁₃, or
(2) when said at least one cationic direct dye is chosen from compounds of formula (III) wherein:
D₁ and D₂ are simultaneously nitrogen atoms,
m is zero, and
E is chosen from E₁, E₂, and E₇,
then said at least one thickening polymer is not chosen from hydroxyalkylcelluloses and carboxyalkylcelluloses.

57. The composition according to claim 56, wherein said keratin fibers are human keratin fibers.

58. The composition according to claim 57, wherein said human keratin fibers are hair.

59. The composition according to claim 56, wherein in formula (I), (II), (III) and (III′), the C₁-C₄ alkyl radicals and the C₁-C₄ alkoxy radicals are chosen from methyl, ethyl, butyl, methoxy and ethoxy radicals.

60. The composition according to claim 56, wherein said anions are chosen from chloride, methyl sulfate and acetate.

61. The composition according to claim 56, wherein said halogen atoms of R₃, R′₃, R₈, R₉, R₁₃, and R₁₅ are chosen from bromine, chlorine, iodine, and fluorine.

62. The composition according to claim 56, wherein said biopolysaccharide gums of microbial origin are chosen from scleroglucan gum and xanthan gum.

63. The composition according to claim 56, wherein said gums derived from plant exudates are chosen from gum arabic, ghatti gum, karaya gum, gum tragacanth, carrageenan gum, agar gum and carob gum.

64. The composition according to claim 56, wherein said at least one cationic direct dye of formula (I) is chosen from compounds of formula (I1) to (I54) below:

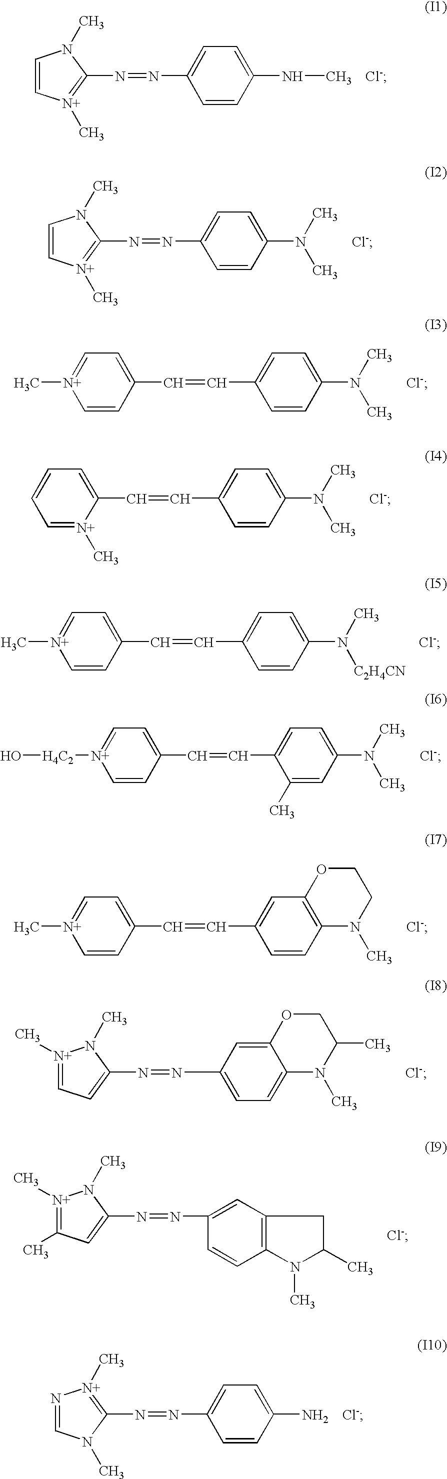

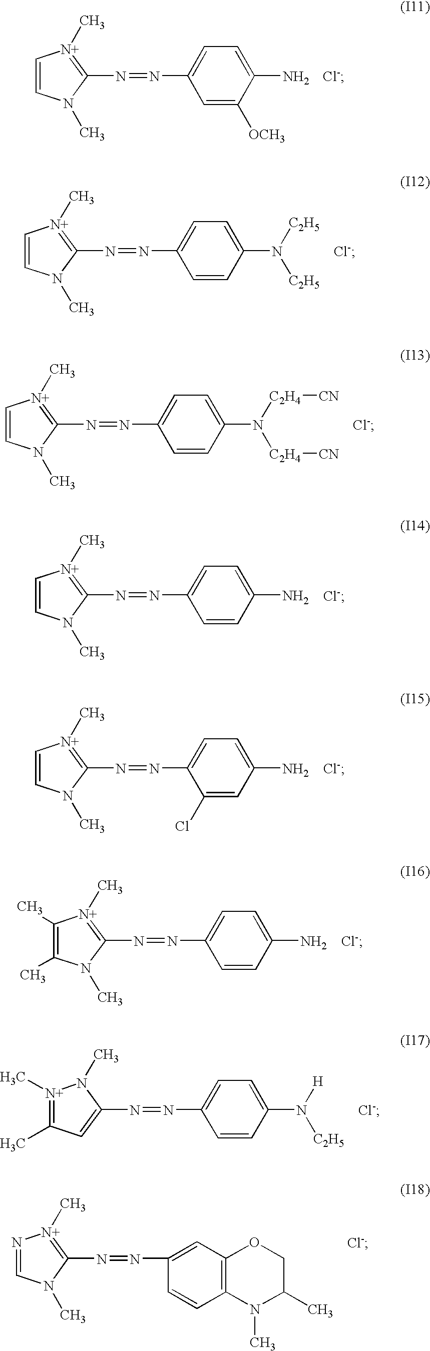

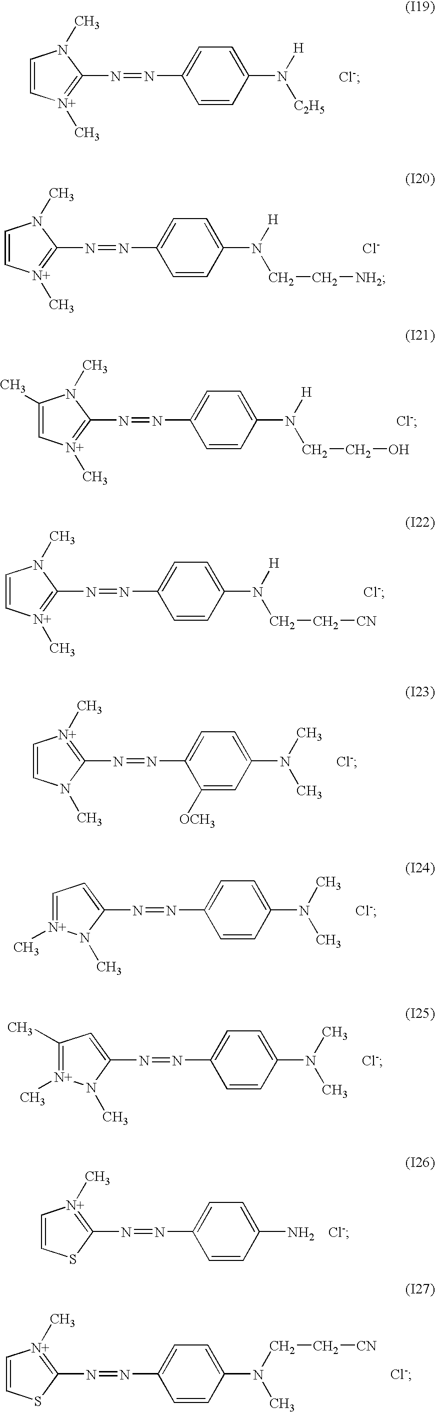

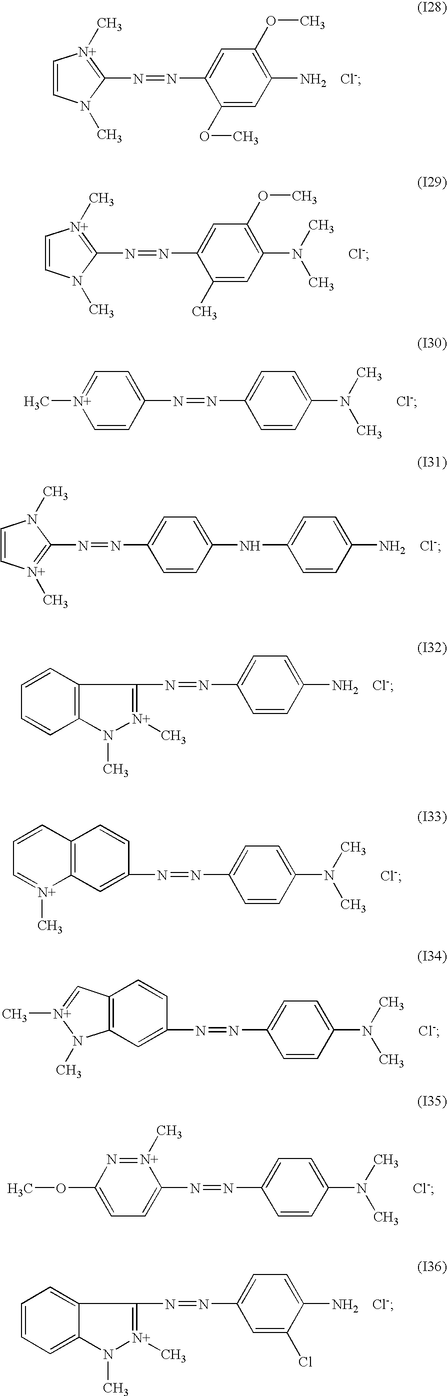

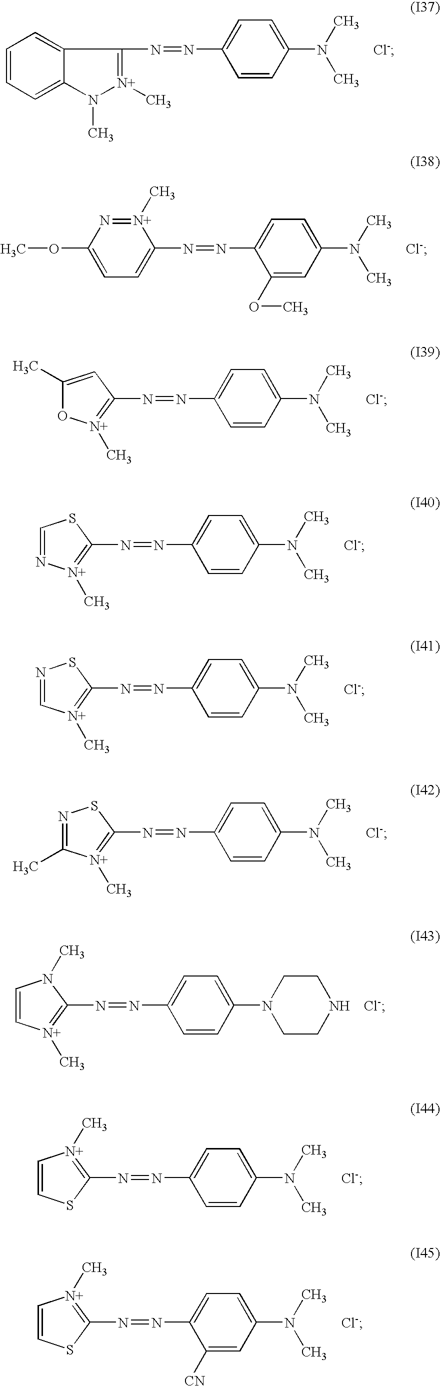

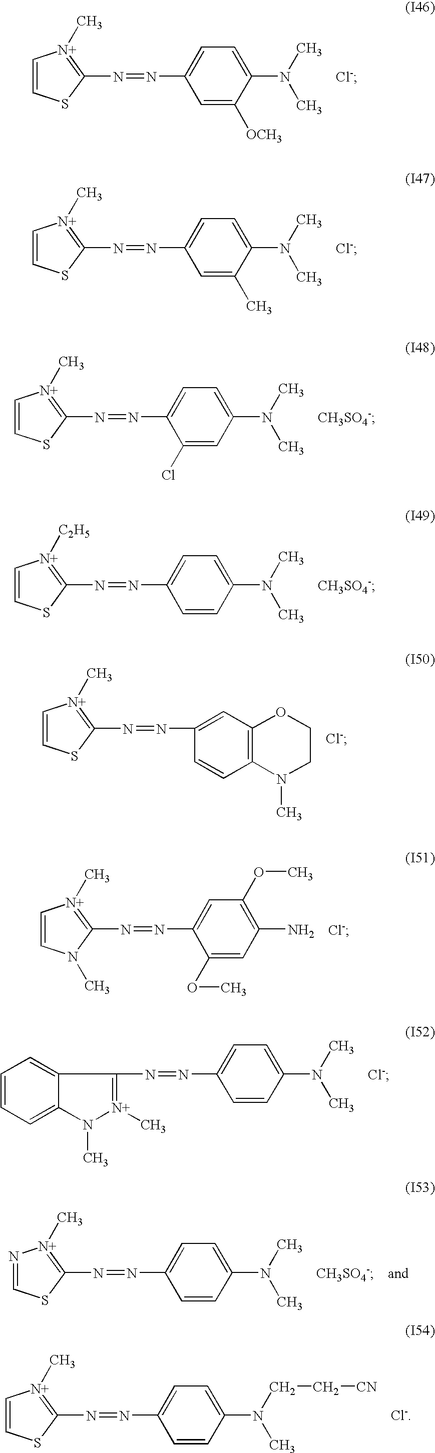

65. The composition according to claim 64, wherein said at least one cationic direct dye is chosen from said compounds of formula (I1), (I2), (I14) and (I31).

66. The composition according to claim 56, wherein said at least one cationic direct dye of formula (II) is chosen from compounds of formula (II1) to (II9) below:

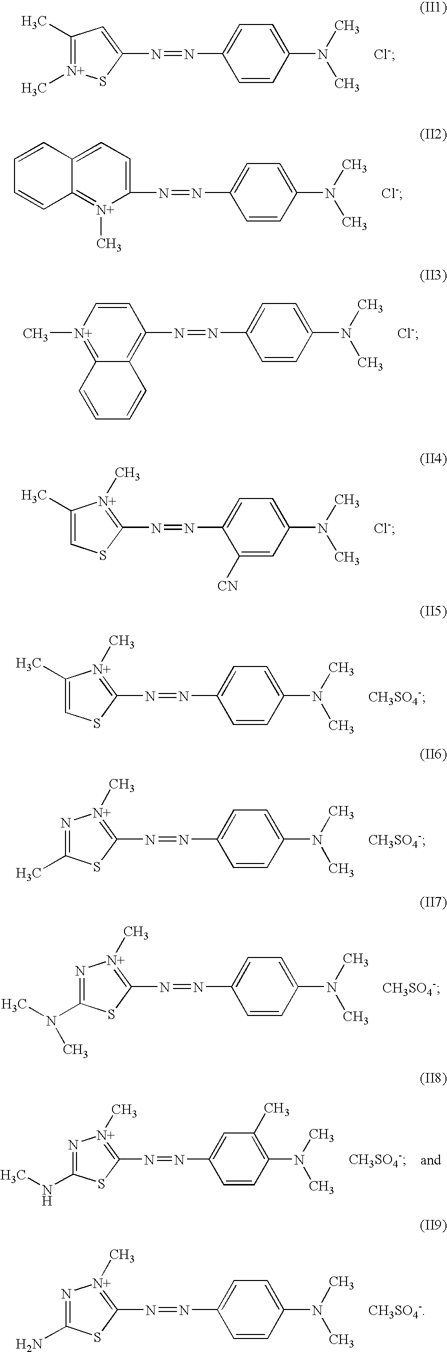

67. The composition according to claim 56, wherein said at least one cationic direct dye of formula (III) is chosen from compounds of formula (III1) to (III18) below:

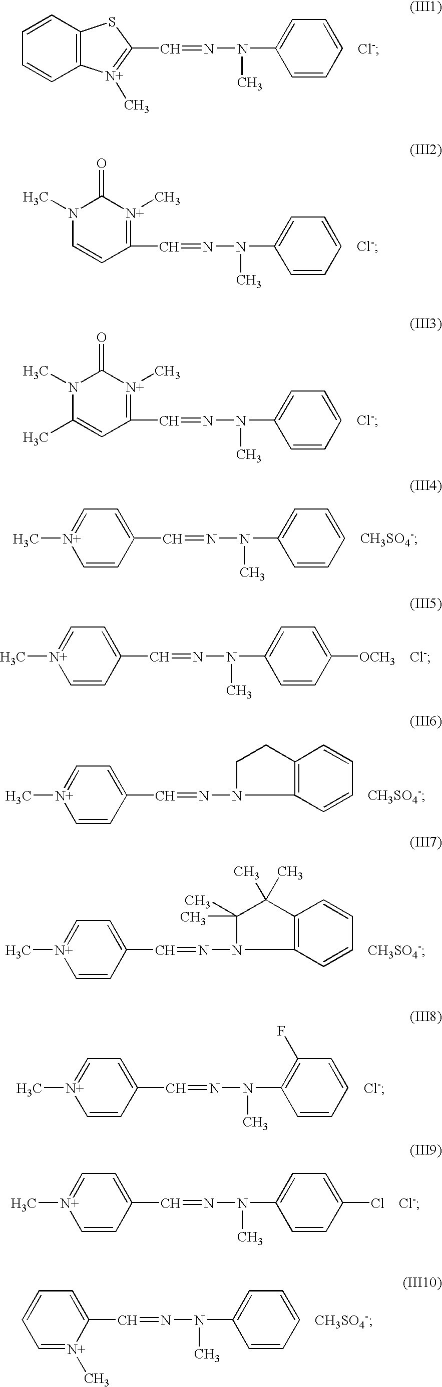

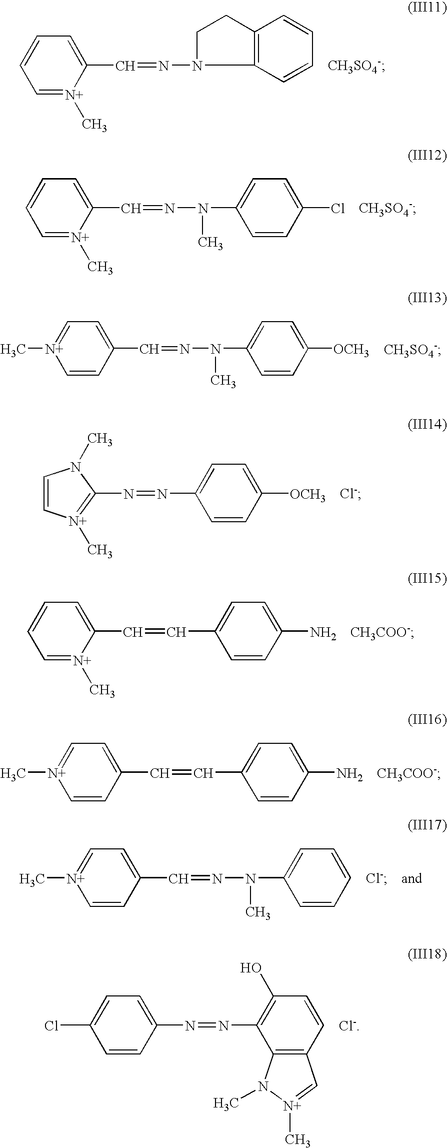

68. The composition according to claim 67, wherein said at least one cationic direct dye of formula (III) is chosen from compounds of formula (III4), (III5) and (III13).

69. The composition according to claim 56, wherein said at least one cationic direct dye of formula (III′) is chosen from compounds of formula (III′1) to (III′3) below:

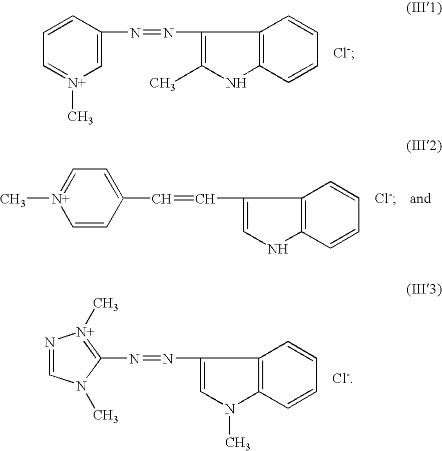

70. The composition according to claim 56, wherein said at least one cationic direct dye of formula (I), (II), (III) or (III′) is present in an amount ranging from 0.001 to 10% by weight relative to the total weight of the composition.

71. The composition according to claim 70, wherein said at least one cationic direct dye of formula (I), (II), (III) or (III′) is present in an amount ranging from 0.005 to 5% by weight relative to the total weight of the composition.

72. The composition according to claim 56, wherein said at least one thickening polymer is chosen from hydroxyalkylcelluloses.

73. The composition according to claim 72, wherein said hydroxyalkylcelluloses are chosen from hydroxyethylcelluloses and hydroxypropylcelluloses.

74. The composition according to claim 56, wherein said at least one thickening polymer is chosen from carboxyalkylcelluloses.

75. The composition according to claim 74, wherein said carboxyalkylcelluloses are carboxymethylcelluloses.

76. The composition according to claim 56, wherein said at least one thickening polymer is a nonionic guar gum modified with C₁-C₆ hydroxyalkyl groups.

77. The composition according to claim 76, wherein said hydroxyalkyl groups are chosen from hydroxymethyl, hydroxyethyl, hydroxypropyl and hydroxybutyl groups.

78. The composition according to claim 76, wherein said nonionic guar gum has a degree of hydroxyalkylation ranging from 0.4 to 1.2.

79. The composition according to claim 56, wherein said at least one thickening polymer is present in an amount ranging from 0.01 to 10% by weight relative to the total weight of the composition.

80. The composition according to claim 79, wherein said at least one thickening polymer is present in an amount ranging from 0.1 to 5% by weight relative to the total weight of the composition.

81. The composition according to claim 56, wherein said composition further comprises a support chosen from water and a mixture of water and at least one organic solvent.

82. The composition according to claim 56, wherein said composition has a pH ranging from 2 to 11.

83. The composition according to claim 82, wherein said composition has a pH ranging from 5 to 10.

84. The composition according to claim 56, wherein said composition further comprises at least one additional direct dye, different from said at least one cationic direct dye (i) as defined in claim 56.

85. The composition according to claim 84, wherein said at least one additional direct dye is chosen from nitrobenzene dyes, anthraquinone dyes, napthaquinone dyes, triarylmethane dyes, xanthene dyes and azo dyes.

86. The composition according to claim 56, wherein said composition further comprises at least one oxidation base chosen from para-phenylenediamines, bis(phenyl)alkylenediamines, para-aminophenols, ortho-aminophenols and heterocyclic bases.

87. The composition according to claim 86, wherein said at least one oxidation base is present in an amount ranging from 0.0005 to 12% by weight relative to the total weight of the dye composition.

88. The composition according to claim 87, wherein said at least one oxidation base is present in an amount ranging from 0.005 to 6% by weight relative to the total weight of the dye composition.

89. The composition according to claim 86, wherein said composition further comprises at least one coupler chosen from meta-phenylenediamines, meta-aminophenols, meta-diphenols and heterocyclic couplers.

90. The composition according to claim 89, wherein said at least one coupler is present in an amount ranging from 0.0001 to 10% by weight relative to the total weight of the dye composition.

91. The composition according to claim 90, wherein said at least one coupler is present in an amount ranging from 0.005 to 5% by weight relative to the total weight of the dye composition.

92. The composition according to claim 86, wherein said composition further comprises at least one oxidizing agent.

93. The composition according to claim 92, wherein said at least one oxidizing agent is chosen from hydrogen peroxide, urea peroxide, alkali metal bromates, persalts and enzymes.

94. The composition according to claim 93, wherein said persalts are chosen from perborates and persulphates.

95. The composition according to claim 93, wherein said enzymes are chosen from peroxidases, lactases, and two-electron oxidoreductases.

96. The composition according to claim 56, wherein said at least one cationic direct dye and said at least one thickening polymer are present in said composition in an amount sufficient for lightening direct dyeing.

97. The composition according to claim 56, wherein said composition further comprises at least one oxidizing agent.

98. The composition according to claim 56, wherein said composition is in a form chosen from a liquid, a shampoo, a cream and a gel.

99. A process for dyeing keratin fibers, comprising applying at least one dye composition to said keratin fibers and developing for a period of time sufficient to achieve a desired coloration, wherein said at least one dye composition comprises:
(i) at least one cationic direct dye chosen from compounds of formula (I), (II), (III) and (III′) below, and
(ii) at least one thickening polymer;
(a) wherein said compounds of formula (I) are chosen from compounds of formula: 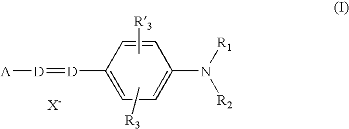
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 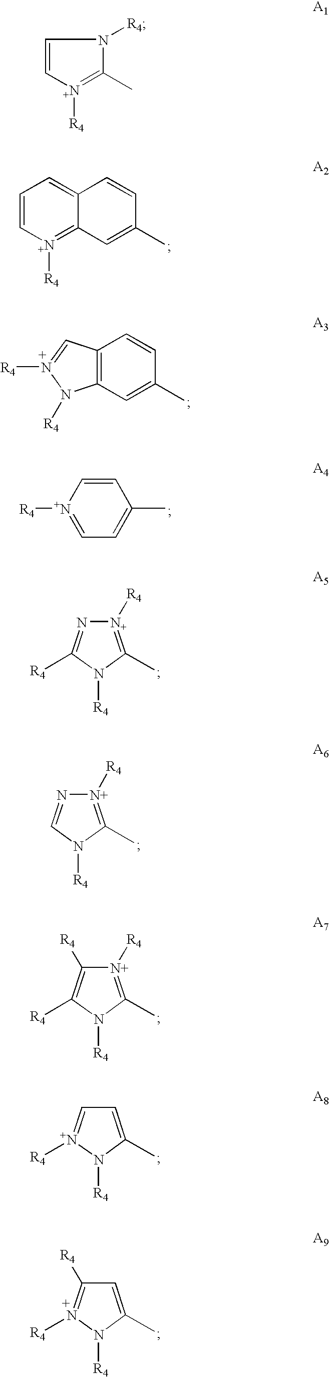 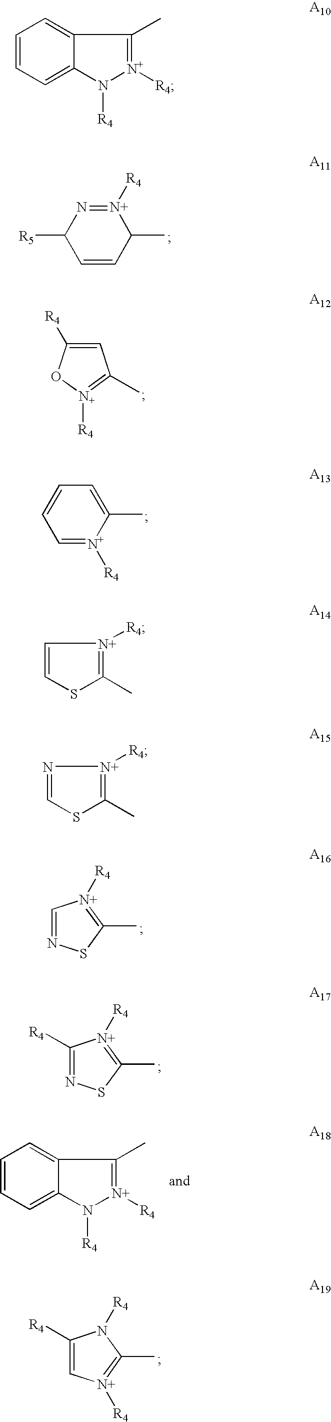
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 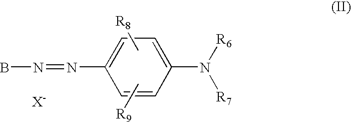
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals, C₁-C₄ alkoxy radicals and a —CN radical,
X⁻ is chosen from anions,
B is chosen from structures B₁ to B₆ below: 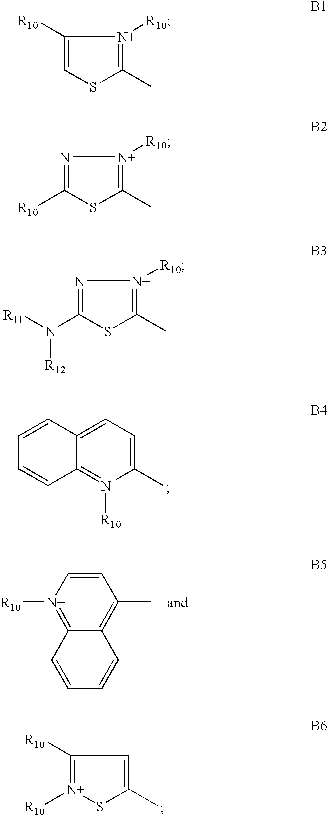
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 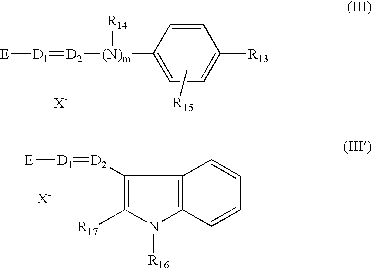
in which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one to radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 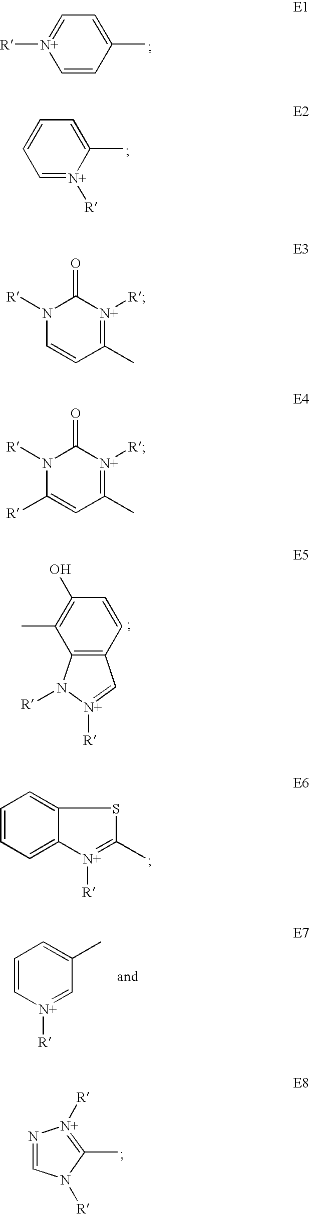
in which R′ is chosen from C₁-C₄ alkyl radicals;
wherein when m is 0 and when D₁ represents a nitrogen atom, E can be further chosen from structure E9 below: 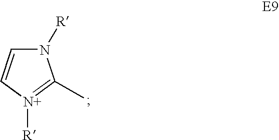
in which R′ is chosen from C₁-C₄ alkyl radicals; and
(d) wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses,
with the provisos that
(1) when said at least one cationic direct dye is chosen from compounds of formula (I) wherein:
both D's are simultaneously nitrogen atoms,
R₃ and R′₃ are simultaneously hydrogen atoms,
R₁ and R₂ are simultaneously unsubstituted methyl radicals, and
A is A₆ wherein R₄ is an unsubstituted methyl radical, or
(2) when said at least one cationic direct dye is chosen from compounds of formula (III) wherein:
D₁ and D₂ are simultaneously nitrogen atoms,
m is zero,
R₁₅ is a hydrogen atom,
R₁₃ is a dimethylamino radical, and
E is E₈ wherein R′ is an unsubstituted methyl group,
then the at least one thickening polymer is not chosen from at least one nonionic guar gum; and
with the further provisos that
(1) when said at least one cationic direct dye is chosen from compounds of formula (I) wherein:
both D's are simultaneously nitrogen atoms, and
A is chosen from A₄ and A₁₃, or
(2) when said at least one cationic direct dye is chosen from compounds of formula (III) wherein:
D₁ and D₂ are simultaneously nitrogen atoms,
m is zero, and
E is chosen from E₁, E₂, and E₇,
then said at least one thickening polymer is not chosen from hydroxyalkylcelluloses and carboxyalkylcelluloses.

100. The process according to claim 99, wherein said process further comprises rinsing said fibers, then drying said fibers.

101. The process according to claim 99, wherein said process further comprises rinsing said fibers, washing said fibers with shampoo, a second rinsing of said fibers and drying of said fibers.

102. A process for dyeing keratin fibers, comprising
separately storing a first composition,
separately storing a second composition,
thereafter mixing said first and second compositions,
applying said mixture to said fibers, and
developing for a period of time sufficient to achieve a desired coloration,
wherein said first composition comprises at least one cationic direct dye chosen from compounds of formula (I), (II), (III) and (III′) below, at least one thickening polymer and at least one oxidation base,
(a) wherein said compounds of formula (I) are chosen from compounds of 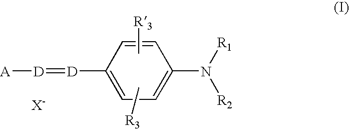
formula:
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 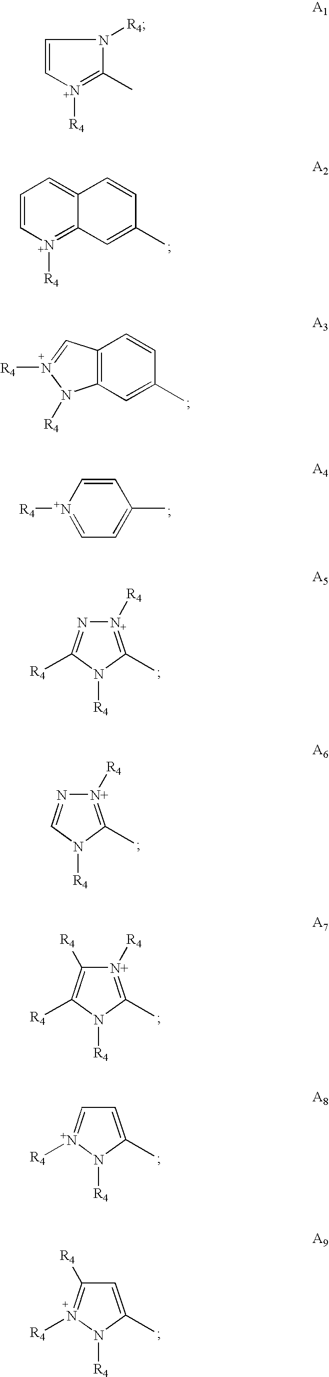 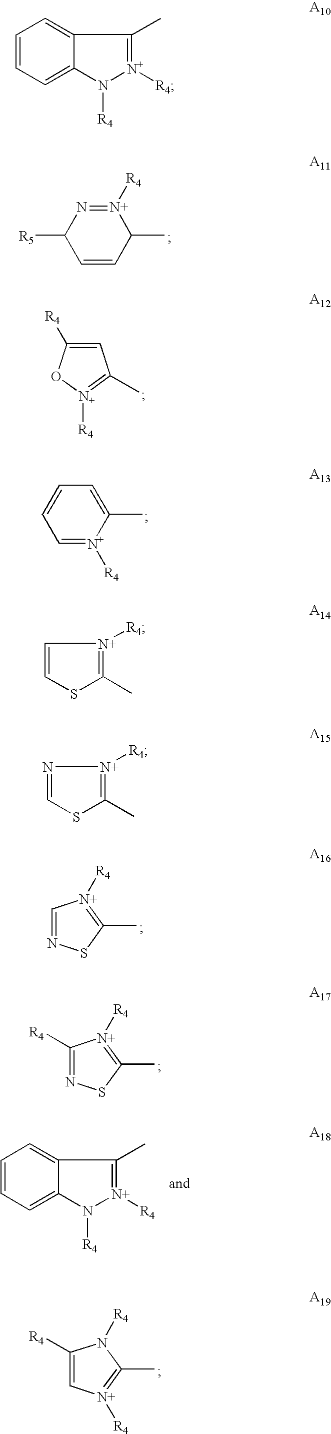
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 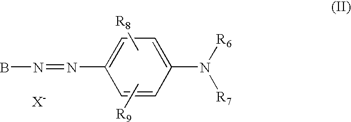
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 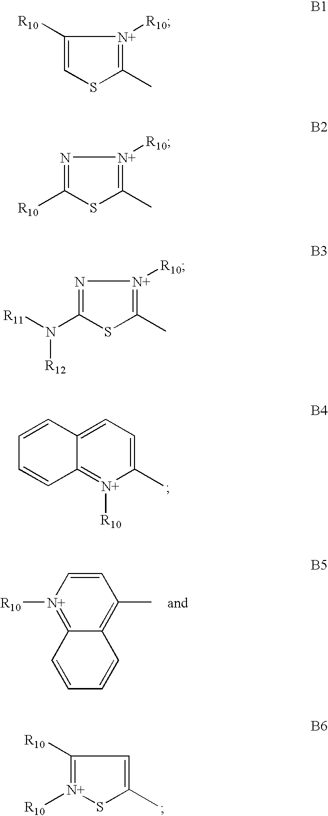
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 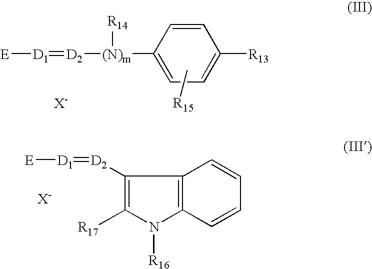
which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 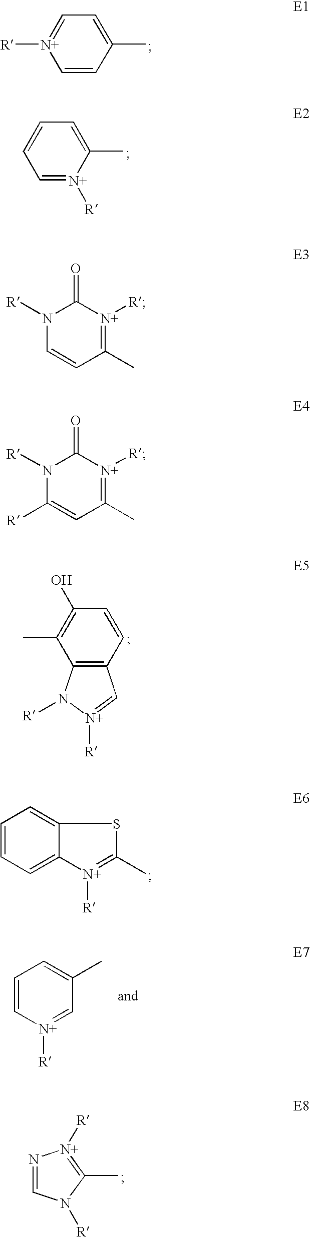
in which R′ is chosen from C₁-C₄ alkyl radicals;
wherein when m is 0 and when D₁ represents a nitrogen atom, E can be further chosen from structure E9 below: 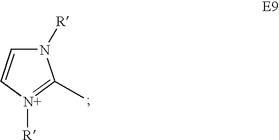
in which R′ is chosen from C₁-C₄ alkyl radicals;
and wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses; and
wherein said second composition comprises at least one oxidizing agent.

103. A process for dyeing keratin fibers, comprising
separately storing a first composition,
separately storing a second composition,
thereafter mixing said first and second compositions,
applying said mixture to said fibers, and
developing for a period of time sufficient to achieve a desired coloration,
wherein said first composition comprises at least one oxidation base, and at least one cationic direct dye chosen from compounds of formula (I), (II), (III) and (III′) below:
(a) wherein said compounds of formula (I) are chosen from compounds of formula: 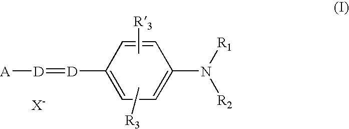
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 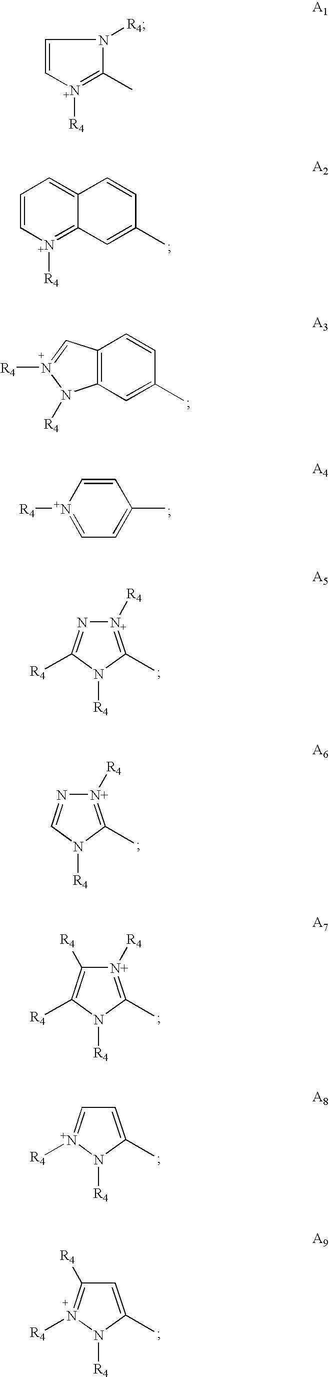 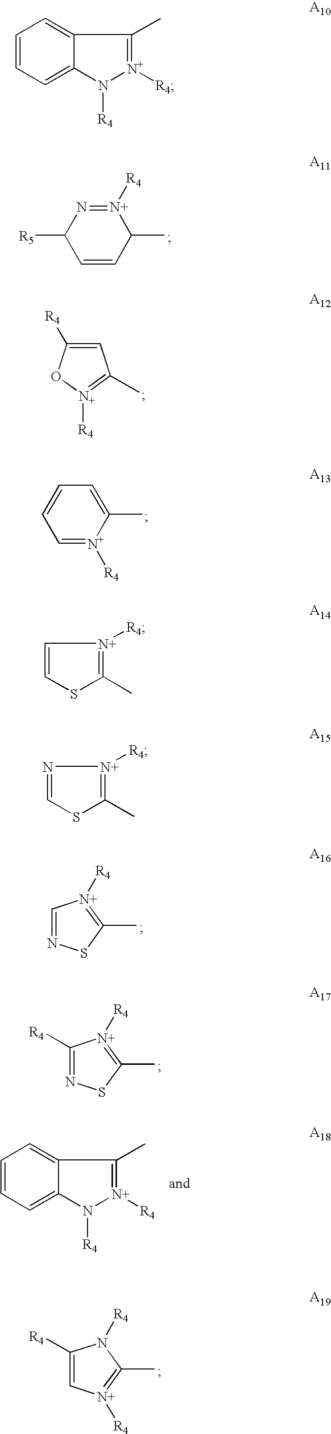
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 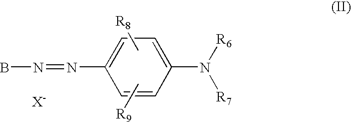
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 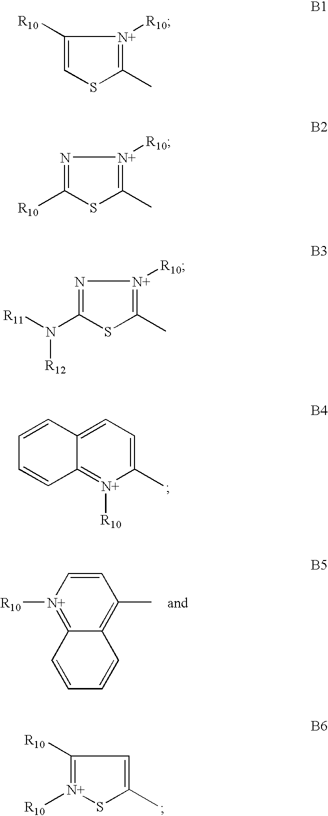
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 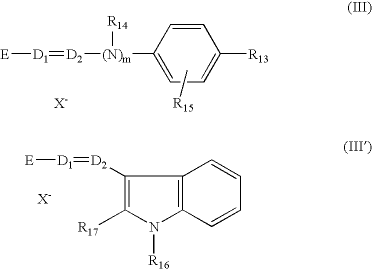
which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 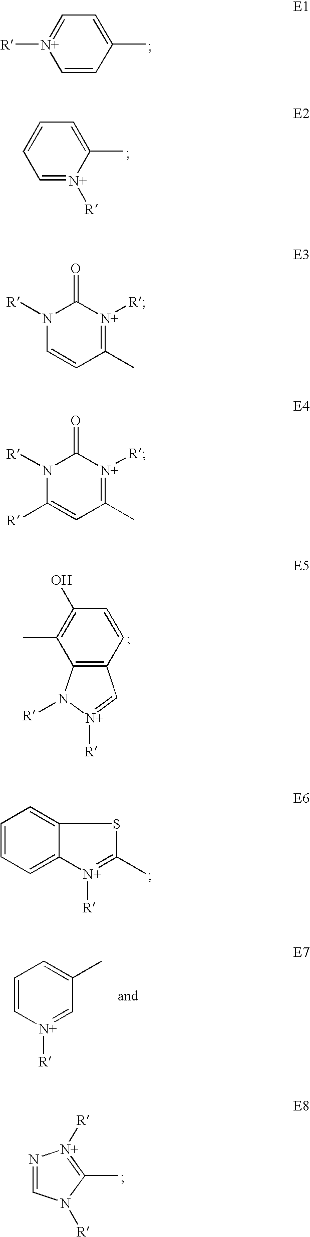
in which R′ is chosen from C₁-C₄ alkyl radicals;
wherein when m is 0 and when D₁ represents a nitrogen atom, E can be further chosen from structure E9 below: 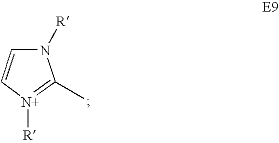
in which R′ is chosen from C₁-C₄ alkyl radicals; and
wherein said second composition comprises at least one oxidizing agent and at least one thickening polymer,
wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses.

104. A process for dyeing keratin fibers, comprising
separately storing a first composition,
separately storing a second composition,
thereafter mixing said first and second compositions,
applying said mixture to said fibers, and
developing for a period of time sufficient to achieve a desired coloration,
wherein said first composition comprises at least one cationic direct dye chosen from compounds of formula (I), (II), (III) and (III′) below and at least one thickening polymer:
(a) wherein said compounds of formula (I) are chosen from compounds of formula: 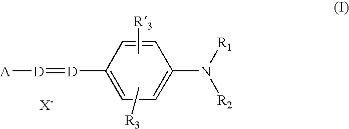
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions, 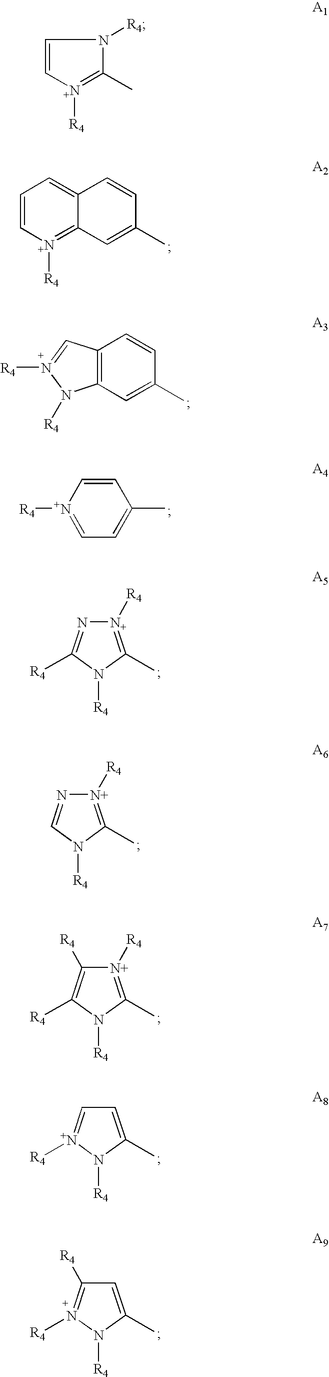 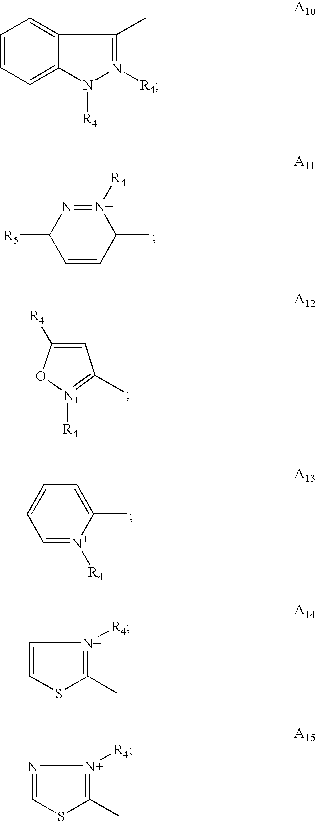
A is chosen from structures A₁ to A₁₉ below: 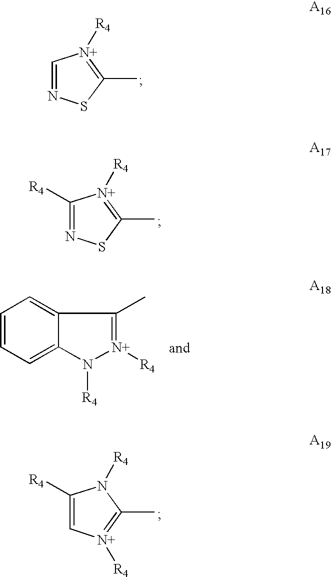
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 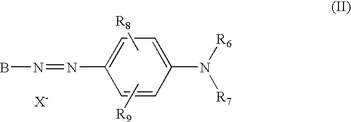
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 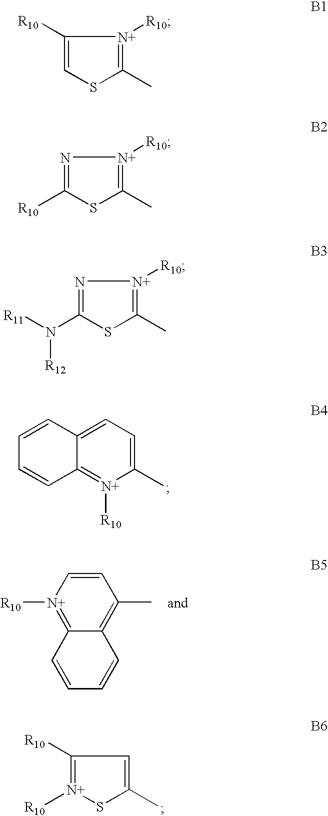
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 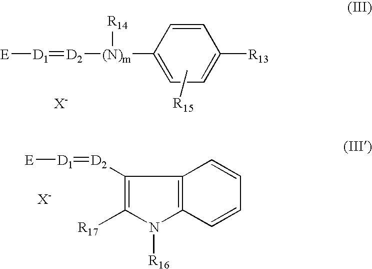
which
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 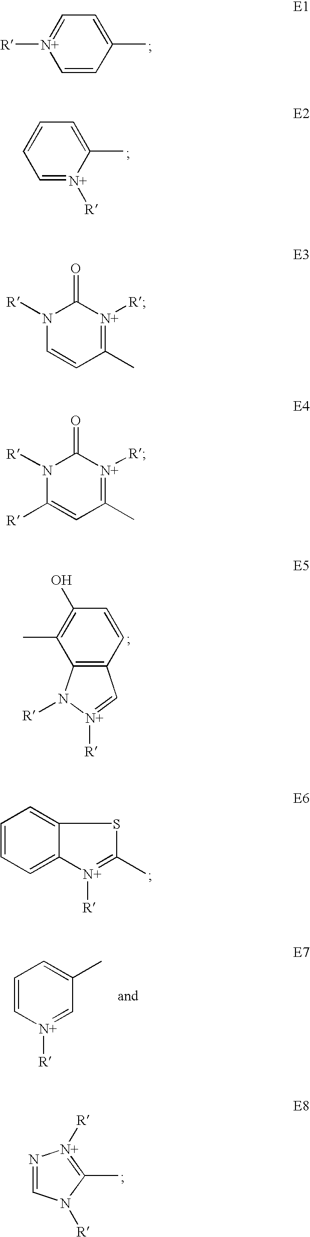
in which R′ is chosen from C₁-C₄ alkyl radicals;
wherein when m is 0 and when D₁ represents a nitrogen atom, E can be further chosen from structure E9 below: 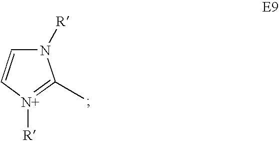
in which R′ is chosen from C₁-C₄ alkyl radicals;
wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses; and
wherein said second composition comprises at least one oxidizing agent.

105. A process for dyeing keratin fibers, comprising
separately storing a first composition,
separately storing a second composition,
thereafter mixing said first and second compositions,
applying said mixture to said fibers, and
developing for a period of time sufficient to achieve a desired coloration,
wherein said first composition comprises at least one cationic direct dye chosen from compounds of formula (I), (II), (III) and (III′) below:
(a) wherein said compounds of formula (I) are chosen from compounds of formula: 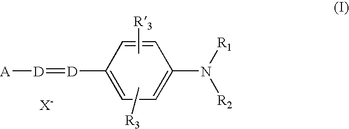
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 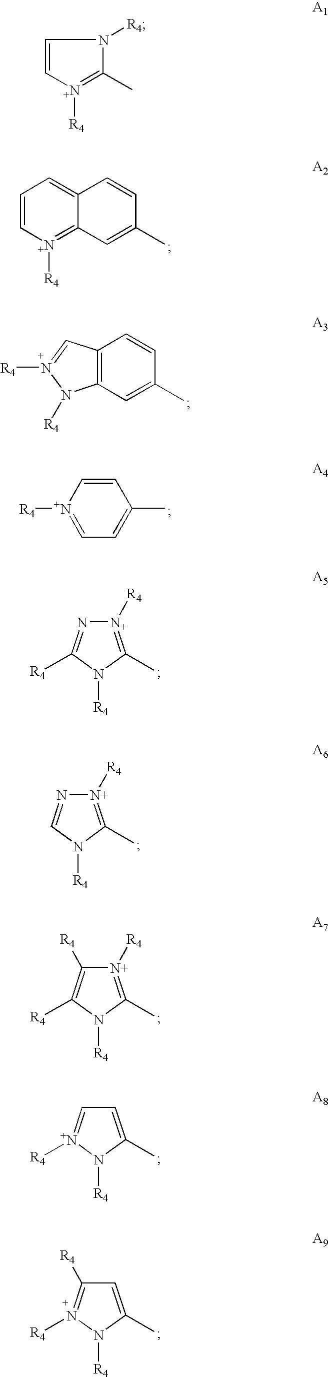 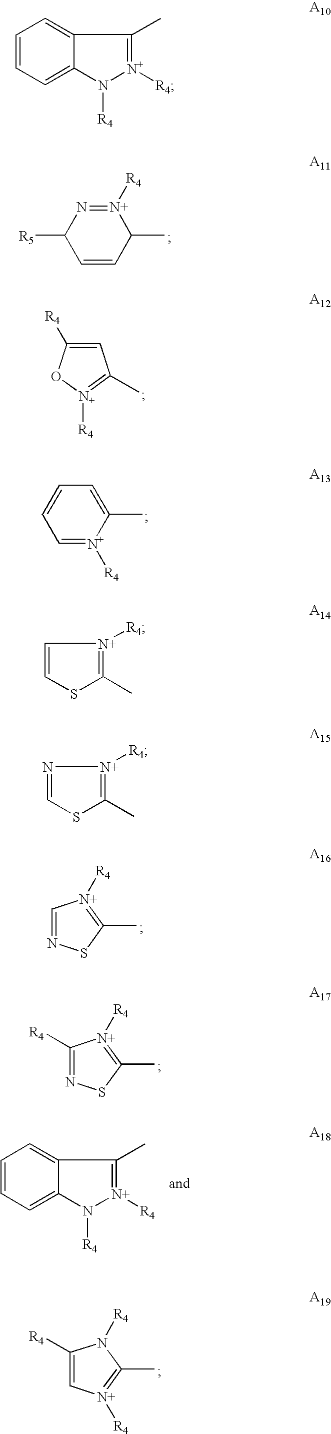
in which:
R₄ is chosen from C₁-C₄ radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 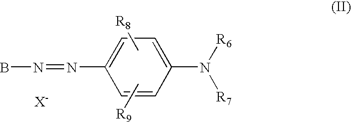
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 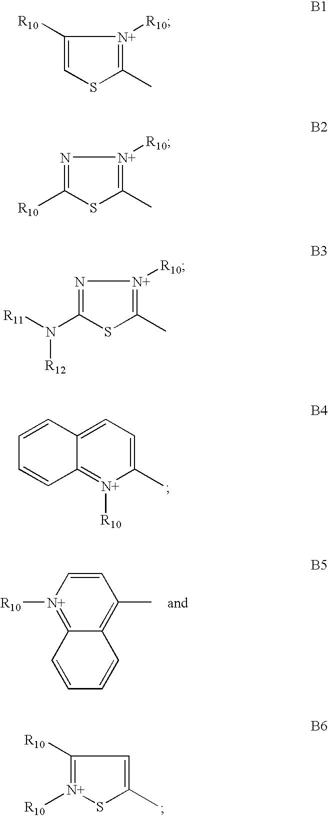
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 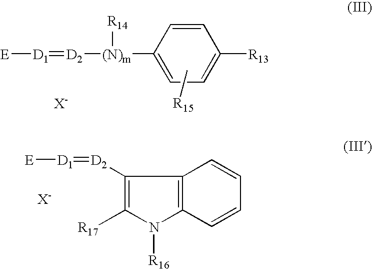
which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 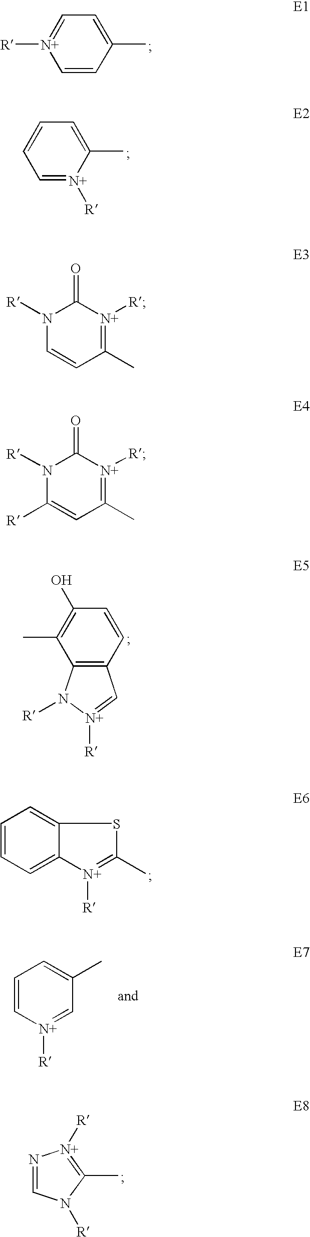
in which R′ is chosen from C₁-C₄ alkyl radicals;
wherein when m is 0 and when D₁ represents a nitrogen atom, E can be further chosen from structure E9 below: 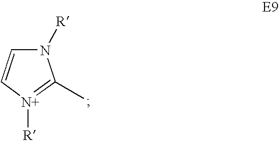
in which R′ is chosen from C₁-C₄ alkyl radicals;
wherein said second composition comprises at least one oxidizing agent and at least one thickening polymer,
wherein said at least one thickening polymer is chosen from:
(ii)₁— nonionic guar gums;
(ii)₂— biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses.

106. A multi-compartment dyeing kit, comprising at least two separate compartments, wherein a first compartment contains a first composition and a second compartment contains a second composition,
wherein said first composition comprises at least one cationic direct dye chosen from compounds of formula (I), (II), (III) and (III′) below, at least one thickening polymer and at least one oxidation base:
(a) wherein said compounds of formula (I) are chosen from compounds of formula: 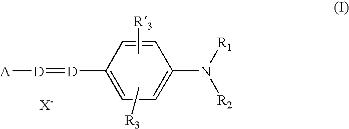
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 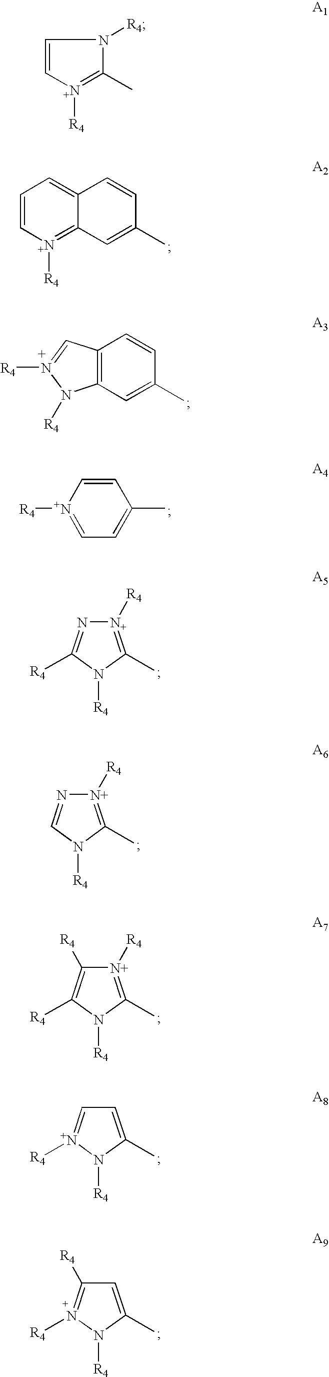 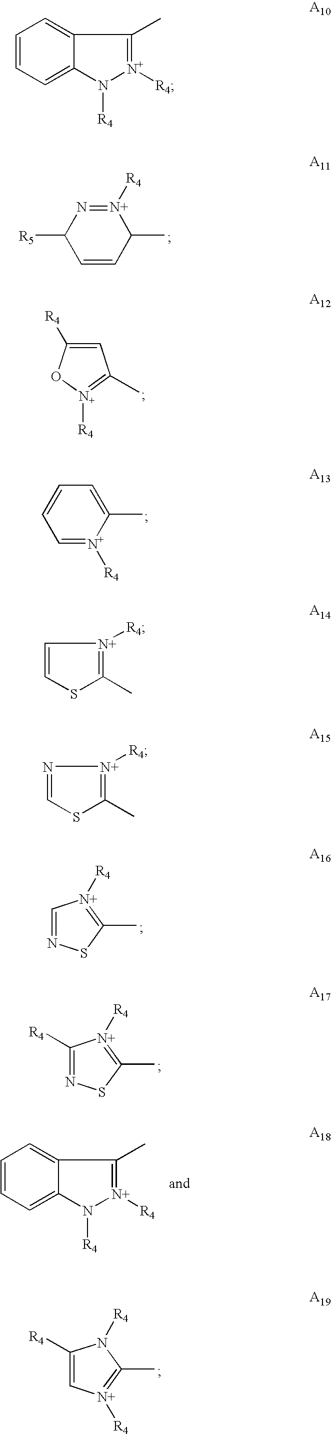
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 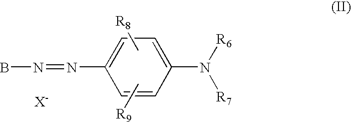
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 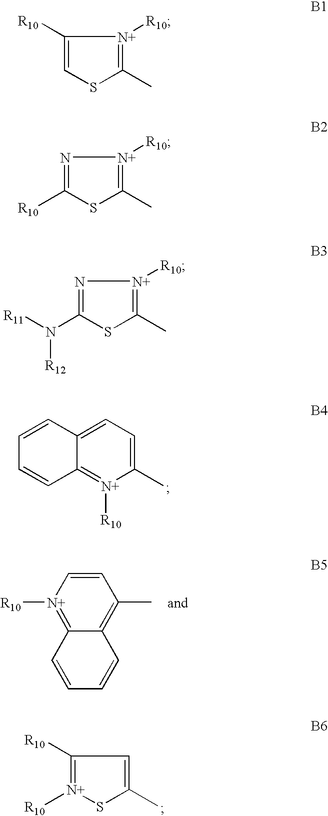
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 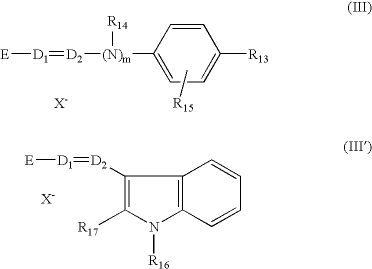
which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 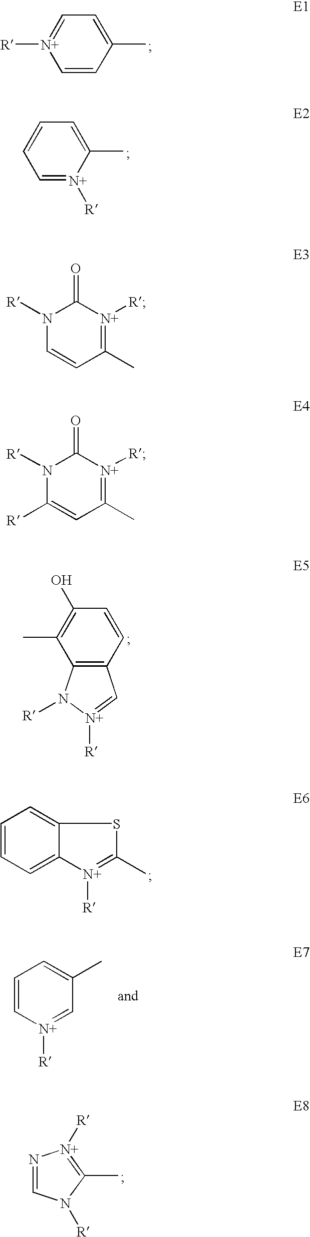
in which R′ is chosen from C₁-C₄ alkyl radicals;
wherein when m is 0 and when D₁ represents a nitrogen atom, E can be further chosen from structure E9 below: 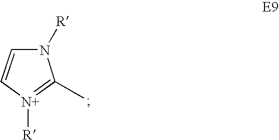
in which R′ is chosen from C₁-C₄ alkyl radicals;
wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses; and
wherein said second composition comprises at least one oxidizing agent.

107. A multi-compartment dyeing kit, comprising at least two separate compartments, wherein a first compartment contains a first composition and a second compartment contains a second composition,
wherein said first composition comprises at least one oxidation base and at least one cationic direct dye chosen from compounds of formula (I), (II), (III) and (III′) below:
(a) wherein said compounds of formula (I) are chosen from compounds of formula: 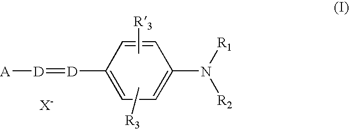
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 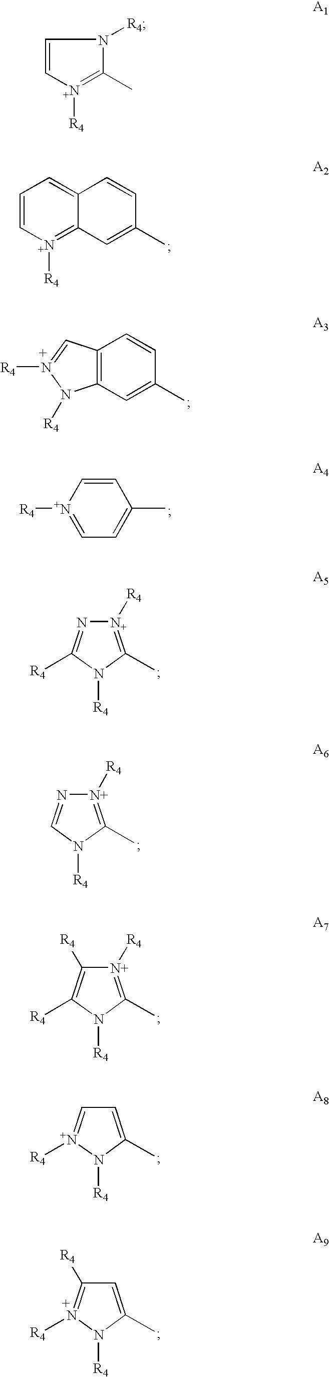 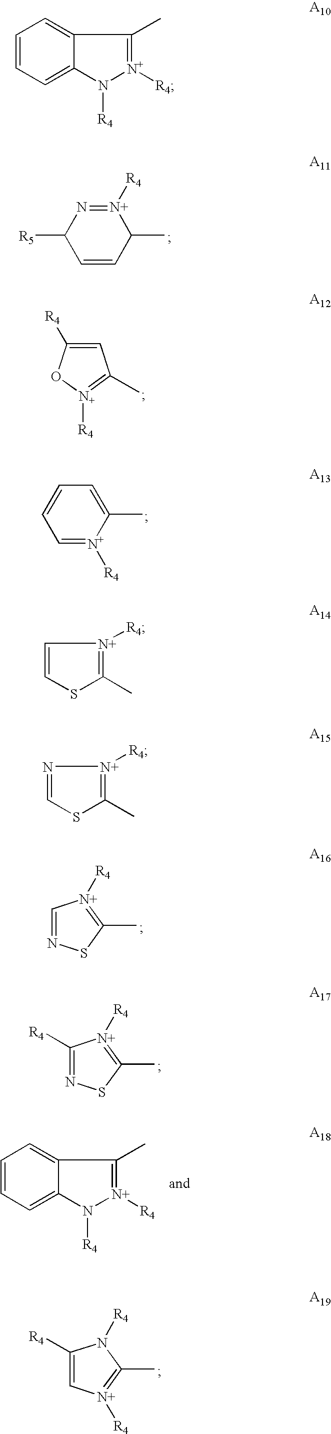
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 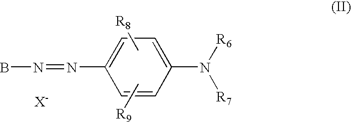
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 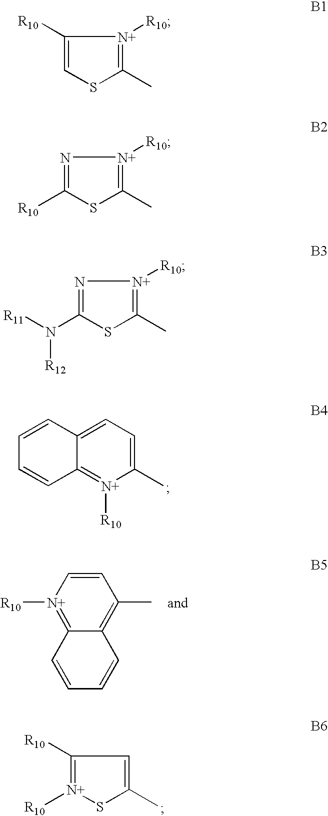
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 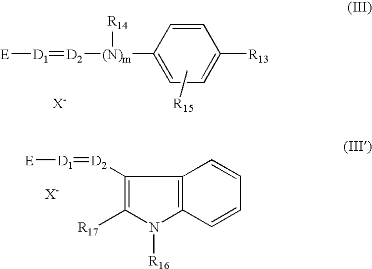
which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 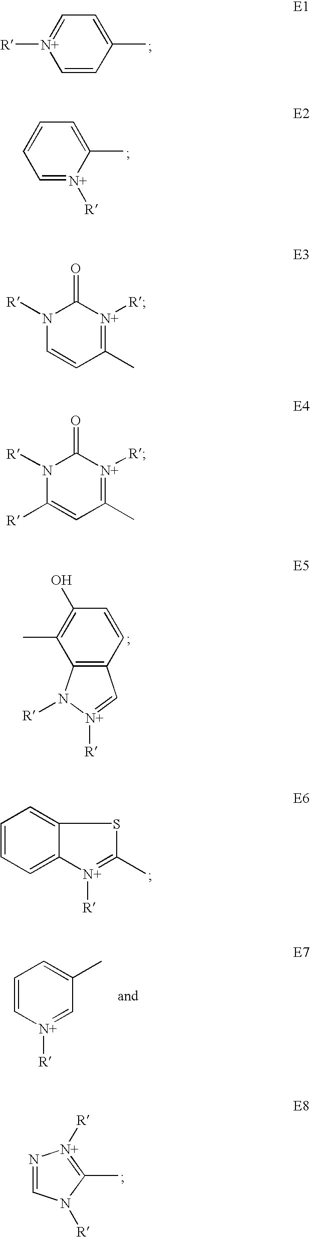
in which R′ is chosen from C₁-C₄ alkyl radicals;
wherein when m is 0 and when D₁ represents a nitrogen atom, E can be further chosen from structure E9 below: 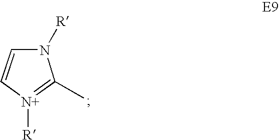
in which R′ is chosen from C₁-C₄ alkyl radicals;
wherein said second composition comprises at least one oxidizing agent and at least one thickening polymer,
wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses.

108. A multi-compartment dyeing kit, comprising at least two separate compartments, wherein a first compartment contains a first composition and a second compartment contains a second composition,
wherein said first composition comprises at least one thickening polymer and at least one cationic direct dye chosen from compounds of formula (I), (II), (III) and (III′) below:
(a) wherein said compounds of formula (I) are chosen from compounds of formula: 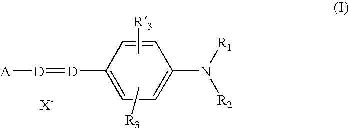
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 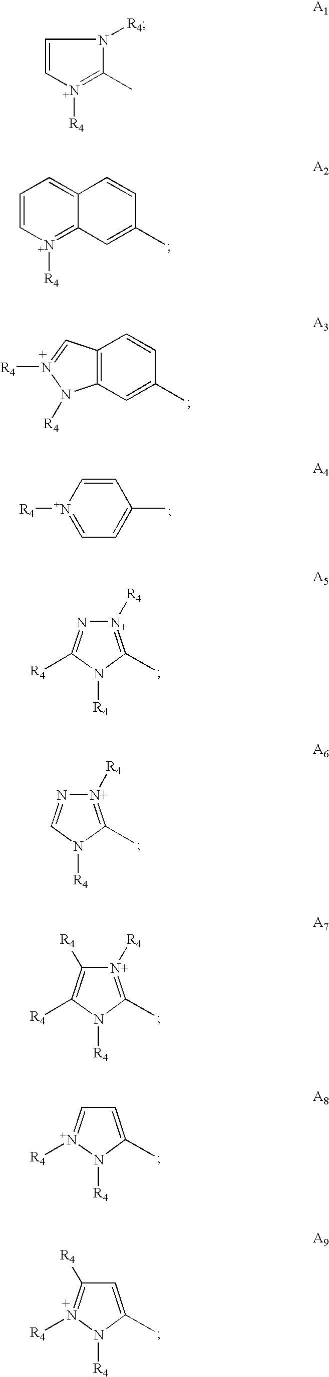 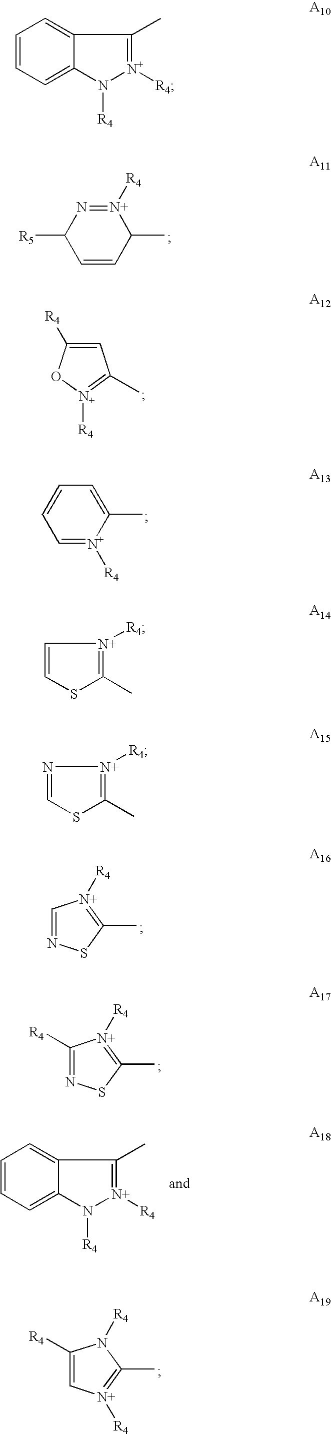
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 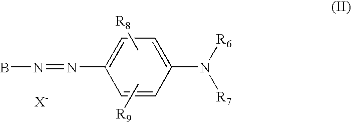
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 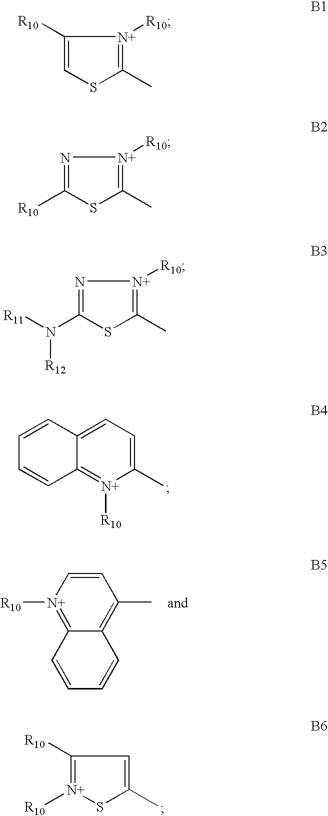
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 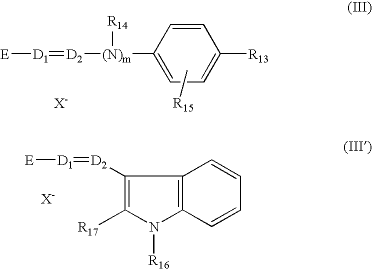
which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 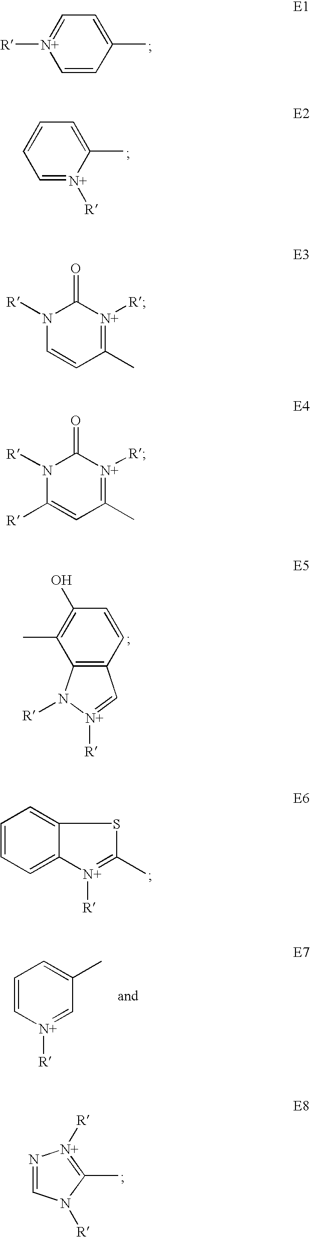
in which R′ is chosen from C₁-C₄ alkyl radicals;
wherein when m is 0 and when D₁ represents a nitrogen atom, E can be further chosen from structure E9 below: 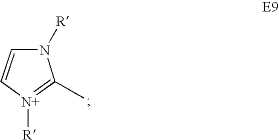
in which R′ is chosen from C₁-C₄ alkyl radicals;
wherein said at least one thickening polymer is chosen from:
(ii)₁— nonionic guar gums;
(ii)₂— biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses; and
wherein said second composition comprises at least one oxidizing agent.

109. A multi-compartment dyeing kit, comprising at least two separate compartments, wherein a first compartment contains a first composition and a second compartment contains a second composition,
wherein said first composition comprises at least one cationic direct dye chosen from compounds of formula (I), (II), (III) and (III′) below:
(a) wherein said compounds of formula (I) are chosen from compounds of formula: 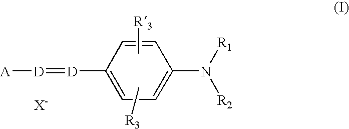
in which:
D is chosen from a nitrogen atom and a —CH group,
R₁ and R₂, which may be identical or different, are chosen from a hydrogen atom; a 4′-aminophenyl radical; and C₁-C₄ alkyl radicals which can optionally be substituted with a radical chosen from —CN, —OH and —NH₂ radicals; or
R₁ and R₂ form, with each other or with a carbon atom of the benzene ring of formula (I), a heterocycle optionally containing a heteroatom chosen from oxygen and nitrogen, which can be substituted with at least one radical chosen from C₁-C₄ alkyl radicals;
R₃ and R′₃, which may be identical or different, are chosen from a hydrogen atom, halogen atoms, a cyano radical, C₁-C₄ alkyl radicals, C₁-C₄ alkoxy radicals and acetyloxy radicals,
X⁻ is chosen from anions,
A is chosen from structures A₁ to A₁₉ below: 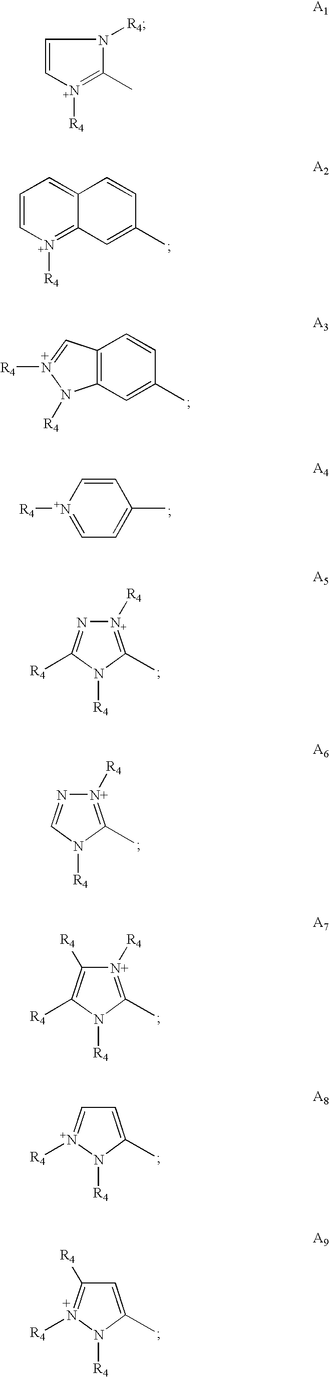 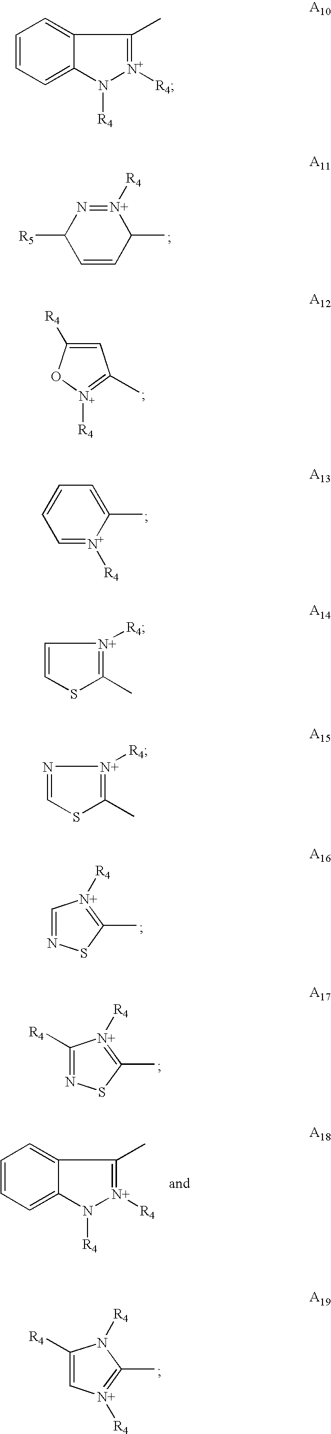
in which:
R₄ is chosen from C₁-C₄ alkyl radicals which can be substituted with a hydroxyl radical, and
R₅ is chosen from C₁-C₄ alkoxy radicals, and
wherein when D represents —CH, when A represents A₄ or A₁₃ and when R₃ is not an alkoxy radical, R₁ and R₂ are not both a hydrogen atom;
(b) wherein said compounds of formula (II) are chosen from compounds of formula: 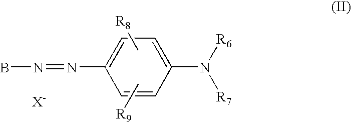
in which:
R₆ is chosen from a hydrogen atom and C₁-C₄ alkyl radicals,
R₇ is chosen from a hydrogen atom, alkyl radicals which can be substituted with a species chosen from a —CN radical and an amino group, and a 4′-aminophenyl radical, or forms, with R₆, a heterocycle optionally comprising at least one heteroatom chosen from oxygen and nitrogen, which can be substituted with C₁-C₄ alkyl radicals,
R₈ and R₉, which may be identical or different, are chosen from a hydrogen atom, halogen atoms, C₁-C₄ alkyl radicals C₁-C₄ alkoxy radicals and a —CN radical,
X⁻ is chosen from anions,
B is chosen from structures B₁ to B₆ below: 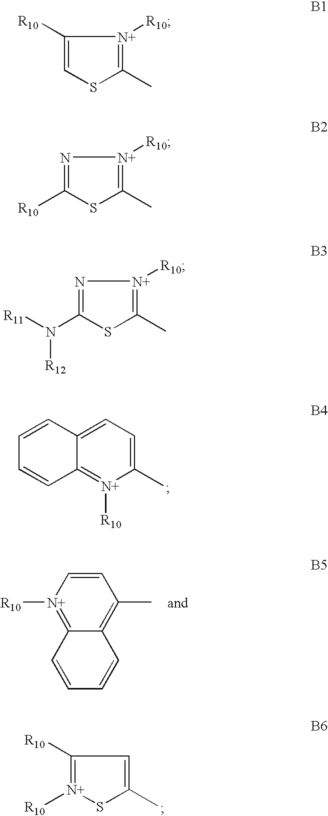
in which:
R₁₀ is chosen from C₁-C₄ alkyl radicals, and
R₁₁ and R₁₂, which may be identical or different, are chosen from a hydrogen atom and C₁-C₄ alkyl radicals;
(c) wherein said compounds of formula (III) and (III′) are chosen from compounds of formula: 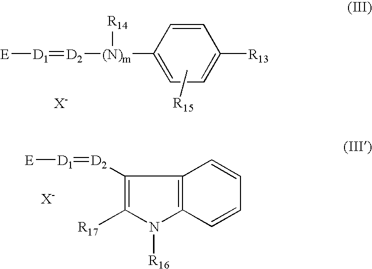
which:
R₁₃ is chosen from a hydrogen atom, C₁-C₄ alkoxy radicals, halogen atoms and an amino radical,
R₁₄ is chosen from a hydrogen atom, C₁-C₄ alkyl radicals or forms, with a carbon atom of the benzene ring, a heterocycle optionally containing an oxygen heteroatom and/or substituted with at least one radical chosen from C₁-C₄ alkyl radicals,
R₁₅ is chosen from a hydrogen atom and halogen atoms,
R₁₆ and R₁₇, which may be identical or different, are chosen from a hydrogen atom and C₁-C₄ alkyl radicals,
D₁ and D₂, which may be identical or different, are chosen from a nitrogen atom and a —CH group,
m is 0 or 1,
wherein when R₁₃ is an unsubstituted amino group, D₁ and D₂ are both a —CH group and m is 0,
X⁻ is chosen from anions,
E is chosen from structures E₁ to E₈ below: 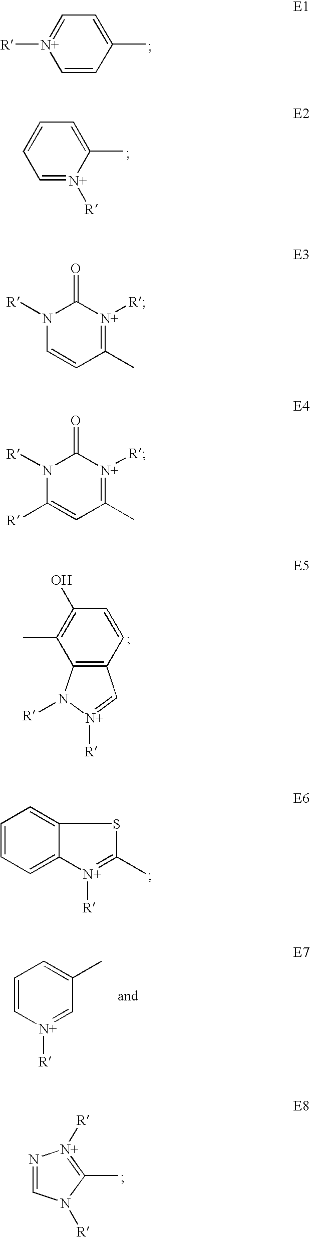
in which R′ is chosen from C₁-C₄ alkyl radicals;
wherein when m is 0 and when D₁ represents a nitrogen atom, E can be further chosen from structure E9 below: 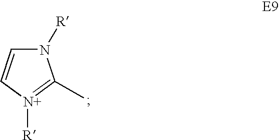
in which R′ is chosen from C₁-C₄ alkyl radicals;
wherein said second composition comprises at least one oxidizing agent and at least one thickening polymer,
wherein said at least one thickening polymer is chosen from:
(ii)₁—nonionic guar gums;
(ii)₂—biopolysaccharide gums of microbial origin;
(ii)₃—gums derived from plant exudates;
(ii)₄—pectins;
(ii)₅—alginates;
(ii)₆—starches; and
(ii)₇—hydroxyalkylcelluloses and carboxyalkylcelluloses.
